SEQUENCE LISTING (10 sequences)

SEQ ID NO: 1 - DNA, 20 bp, synthetic construct
  source 1..20: mol_type = other DNA
aaacccatattggatgctaa

SEQ ID NO: 2 - DNA, 24 bp, synthetic construct
  source 1..24: mol_type = other DNA
aaacccatattggatgctaactag

SEQ ID NO: 3 - DNA, 24 bp, synthetic construct
  source 1..24: mol_type = other DNA
aaacccatattggatgctaatagc

SEQ ID NO: 4 - DNA, 24 bp, synthetic construct
  source 1..24: mol_type = other DNA
aaacccatattggatgctaagcta

SEQ ID NO: 5 - DNA, 24 bp, synthetic construct
  source 1..24: mol_type = other DNA
aaacccatattggatgctaaagct

SEQ ID NO: 6 - DNA, 15 bp, synthetic construct
  source 1..15: mol_type = other DNA
tataacctacgatta

SEQ ID NO: 7 - DNA, 15 bp, synthetic construct
  source 1..15: mol_type = other DNA
tataacctacgattc

SEQ ID NO: 8 - DNA, 15 bp, synthetic construct
  source 1..15: mol_type = other DNA
tataacctacgattg

SEQ ID NO: 9 - DNA, 15 bp, synthetic construct
  source 1..15: mol_type = other DNA
tataacctacgattt

SEQ ID NO: 10 - DNA, 18 bp, synthetic construct
  source 1..18: mol_type = other DNA
tataacctacgatttcga

(19) United States
(12) Patent Application Publication
(10) Pub. No.: US 2025/0250632 A1
(43) Pub. Date: Aug. 7, 2025

(54) MOLECULAR BARCODE READERS FOR ANALYTE DETECTION

(71) Applicant: 10x Genomics, Inc., Pleasanton, CA (US)

(72) Inventors: Marco Mignardi, Stockholm (SE); Marco Serra, Dublin, CA (US); David Hoffman, Pleasanton, CA (US); Xiaoyan Qian, Stockholm (SE)

(21) Appl. No.: 18/855,514

(22) PCT Filed: May 5, 2023
(86) PCT No.: PCT/US2023/021146

§ 371 Date: Oct. 9, 2024

Related U.S. Application Data
(60) Provisional application No. 63/339,237, filed on May 6, 2022.

(51) Int. Cl. C12Q 1/6876; C12Q 1/6841

(52) U.S. Cl. CPC C12Q 1/6876; C12Q 1/6841; C12Q 2600/16

(57) ABSTRACT

Provided herein are methods of detecting a target nucleic acid of interest for spatial gene expression analysis in a sample using barcoded templated ligation probes.

### CROSS-REFERENCE TO RELATED APPLICATION

This application claims priority to U.S. Provisional Patent Application No. 63/339,237, filed May 6, 2022. This application is incorporated herein by reference in its entirety.

### REFERENCE TO SEQUENCE LISTING

This application contains a Sequence Listing that has been submitted electronically as an XML file named 47706_0327WO1_SL_ST26.xml. The XML file, created on May 4, 2023, is 9,790 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Various methods exist for profiling the identity, abundance, and location of analytes within a tissue, including array-based and sequencing-based methods. Current in situ hybridization and sequencing-based approaches suffer from low efficiency but the potential value of such in-tissue analysis is significant. End-point readout of spatial analysis methods is a critical step in generating robust data for either targeted or whole-transcriptome profiling of analytes within cells of a tissue, with resolution, throughput, and reagent costs being significant factors. Therefore, there exists a need for new and improved methods for reading out end-point data for spatial analysis of analytes within cells of a tissue.

### SUMMARY

Targeted RNA capture is an attractive alternative to poly(A) mRNA capture for interrogating spatial gene expression in a sample (e.g., an FFPE tissue). Compared to poly(A) mRNA capture, targeted RNA capture as described herein is less affected by RNA degradation associated with FFPE fixation compared to methods dependent on capture of polyadenylated mRNA and reverse transcription of mRNA. Targeted RNA capture as described herein allows for sensitive measurement of specific genes of interest that otherwise might be missed with a whole transcriptomic approach. Targeted RNA capture can be used to capture a defined set of RNA molecules of interest, or it can be used at a whole transcriptome level, or anything in between.

After templated ligation, also referred to as RNA-templated ligation (RTL), capture of ligation products on the array and extension of capture probes, several methods exist for detecting the capture of the targeted sequences. When using barcoded templated ligation probes, unique combinations of the one or more barcodes of the first probe and the one or more barcodes of the second probe can be determined and can specifically identify the RNA analyte to which the first and second targeted probes hybridized. Barcoded probes can be detected on the array by microscopy, for example, fluorescent microscopy, interferometric cross-polarization microscopy, or electron microscopy.

If a single channel of detection is used to detect unique combinations of barcodes, combinatorial 2-bit signatures identifying target analytes can be read out using cycles of annealing and combinatorial detection. Imaging one channel, for example by microscopy, is advantageously fast, and the signal for one-channel detection of beads or nanoparticles is advantageous in one plane on the array. The method of one-channel 2-bit detection also advantageously has very low background signal. Further, one-channel detection of hybridized oligonucleotide conjugates does not require amplification, saving time and reagent costs.

In one aspect, provided herein is method for identifying the location of a target nucleic acid in a biological sample. In some instances, the method includes (a) providing the biological sample on an array including a plurality of capture probes, where a capture probe of the plurality of capture probes includes a capture domain; (b) contacting a first probe and a second probe within the biological sample, where the first probe and the second probe each include sequences that are substantially complementary to sequences of the target nucleic acid, where the first probe or the second probe include a capture probe capture domain that is complementary to all or a portion of the capture domain on the array, where the first probe and/or the second probe include one or more barcode sequences selected from a plurality of barcode sequences, such that a combination of the first probe and the second probe includes a unique combination of the one or more barcode sequences; (c) hybridizing the first probe and the second probe to the target nucleic acid; (d) generating a ligation product by ligating the first probe and the second probe; (e) releasing the ligation product from the target nucleic acid; (f) hybridizing the ligation product to the capture domain on the array; and (g) decoding the one or more barcode sequences or complements thereof, and using the decoded one or more barcode sequences to identify the location of the target nucleic acid in the biological sample.

In some implementations, the first probe and the second probe are DNA probes. In some implementations, the first probe and/or the second probe include 2, 3, 4, 5, or more barcode sequences selected from the plurality of barcode sequences. In some implementations, the ligation product includes the combination of the one or more barcode sequences. In some implementations, the combination of the one or more barcode sequences identifies the target nucleic acid.

In some implementations, the method further includes: (a) a first probe including, in order from 5′ to 3′: (i) a functional sequence, (ii) one or more barcode sequences, and (iii) a sequence substantially complementary to at least a portion of the sequence of the target nucleic acid; and/or (b) a second probe including, in order from 5′ to 3′: (i) a sequence substantially complementary to at least a portion of the sequence of the target nucleic acid, (ii) one or more barcode sequences, and (iii) a capture probe capture domain.

In some implementations, the decoding step further includes steps of (i) extending the capture domain using the ligation product as a template; (ii) hybridizing a first primer to a first barcode sequence of the plurality of barcode sequences of the extended capture domain, wherein the first primer is conjugated to a labeling agent, and wherein the first primer includes a sequence substantially complementary to the first barcode sequence; (iii) detecting hybridization of the first primer to the first barcode sequence; (iv) removing the first primer from the first barcode sequence; and (v) repeating steps (ii) through (v) using a second, a third, a fourth, a fifth, or more primers, each including the labeling agent, to detect a second, a third, a fourth, a fifth, or more barcode sequences, respectively.

In some implementations, the labeling agent is a bead. In some implementations, the labeling agent is a nanoparticle. In some implementations, the labeling agent is a gold nanoparticle. In some implementations, the detecting hybridization of the first primer includes imaging the array. In some implementations, imaging the array is by electron microscopy. In some implementations, imaging the array is by interferometric cross-polarization microscopy.

In some implementations, the decoding step further includes: (i) extending the capture domain using the ligation product as a template, (ii) hybridizing a first primer to a first barcode sequence of the plurality of barcode sequences of the extended capture domain, wherein the first primer includes a sequence substantially complementary to the first barcode sequence; (iii) applying a mixture of dNTPs and a polymerase to the array; (iv) extending the first primer to produce a first extension product; (v) detecting the first extension product; (vi) repeating steps (ii) through (v) using a second, a third, a fourth, a fifth, or more primers to detect a second, a third, a fourth, a fifth, or more extension products, respectively.

In some implementations, each of the first, the second, the third, the fourth, the fifth, or more primers hybridize to unique barcode sequences of the plurality of barcodes sequences. In some implementations, the mixture of dNTPs comprises dATP, dTTP, dCTP, and dGTP. In some implementations, one of dATP, dTTP, dCTP or dGTP is labeled with a fluorophore. In some implementations, for each cycle of steps (ii) through (v), the dATP, dTTP, dCTP or dGTP is labeled with a unique fluorophore. In some implementations, the detecting comprises imaging the array. In some implementations, imaging the array is by fluorescent microscopy. In some implementations, the first primer is selected from a set of four primers, each primer having a different 3′-terminal nucleotide, the first primer having a 3′-terminal nucleotide complementary to the first 5′ nucleotide of the sequence that is substantially complementary to the sequence of the target nucleic acid. In some implementations, the set of four primers each include one or more degenerate nucleotide positions at 3′ end of the primer.

In some implementations, the first probe includes a sequence that is substantially complementary to a first sequence of the target nucleic acid and the second probe includes a sequence that is substantially complementary to a second sequence of the target nucleic acid. In some implementations, the first sequence and second sequence are adjacent to one another on the target nucleic acid. In some implementations, ligating the first probe and the second probe utilizes a ligase. In some implementations, the ligase is T4 DNA ligase, or PBCV-1 ligase or an equivalent thereof. In some implementations, the method further includes applying a reagent medium to the biological sample on the array, wherein the reagent medium comprising a permeabilization agent. In some implementations, the reagent medium includes an agent for releasing the ligation product, with the agent for releasing the ligation product including a nuclease. In some implementations, the nuclease includes an RNase, optionally with the RNase selected from RNase A, RNase C, RNase H, or RNase I. In some implementations, the permeabilization agent includes a protease. In some implementations, the protease is selected from trypsin, pepsin, elastase, or proteinase K. In some implementations, the reagent medium further includes a detergent. In some implementations, the detergent is selected from sodium dodecyl sulfate (SDS), sarkosyl, saponin, Triton X-100™, or Tween-20™. In some implementations, the reagent medium includes less than 5 w/v % of a detergent selected from SDS and sarkosyl. In some implementations, the reagent medium includes at least 5% w/v % of a detergent selected from SDS and sarkosyl. In some implementations, the reagent medium does not include sodium dodcyl sulfate (SDS) or sarkosyl. In some implementations, the biological sample on the array is contacted with the reagent medium for about 1-60 minutes. In some implementations, the biological sample on the array is contacted with the reagent medium for about 30 minutes.

In some implementations, the target nucleic acid is an RNA. In some implementations, the RNA is an mRNA. In some implementations, the capture domain includes a poly(T) sequence. In some implementations, the capture domain includes a fixed sequence. In some implementations, the capture domain includes a spatial barcode. In some implementations, the biological sample is a tissue sample or a cell culture sample. In some implementations, the tissue sample is a solid tissue sample. In some implementations, the solid tissue sample is a tissue section. In some implementations, the tissue sample is a fixed tissue sample. In some implementations, the fixed tissue sample is a formalin fixed paraffin embedded (FFPE) tissue sample. In some implementations, the FFPE tissue is deparaffinized and decrosslinked. In some implementations, the fixed tissue sample is a formalin fixed paraffin embedded cell pellet. In some implementations, the tissue sample is a fresh frozen tissue sample. In some implementations, the tissue sample is fixed and stained. In some implementations, the tissue sample is stained using immunofluorescence, immunohistochemistry, or using a hematoxylin and eosin (H&E) stain.

In any one of the methods disclosed herein, the array can be on the first substrate.

In any one of the methods disclosed herein, the array can be on a second substrate. In any one of the methods disclosed herein, the first substrate is aligned with the second substrate, such that at least a portion of the biological sample is aligned with at least a portion of the array on the first substrate.

In another aspect, provided herein is a composition comprising: (a) an array including capture probes, wherein the capture probes include a capture domain; (b) a biological sample on the array, where the biological sample includes a plurality of target nucleic acids of interest; and (c) a first probe and a second probe hybridized to a target nucleic acid and ligated together, where the first probe and the second probe each include a sequence that is substantially complementary to adjacent sequences of the target nucleic acid, where the first probe and/or the second probe contain one or more unique barcode sequences selected from a plurality of barcode sequences, and where one of the first probe or the second probe comprises a capture probe capture domain.

In some implementations, the first probe and second probe are ligated together and collectively include a unique combination of barcode sequences that identify the analyte. In some implementations, the composition further includes a first primer that is hybridized to one of the barcode sequences, where the first primer is conjugated to a labeling agent. In some implementations, the composition further includes a first primer that is hybridized to one of the barcode sequences and a mixture of dNTPs, wherein the mixture of dNTPs comprises at least one dNTP that is labeled with a fluorophore.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for a particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in a set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing a templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 shows an exemplary spatial analysis workflow using barcoded probes for templated ligation.

FIG. 13 is a schematic diagram showing exemplary barcoded probes for templated ligation.

FIG. 14 is a schematic diagram showing barcoded probes hybridized to a template nucleic acid within a biological sample, followed by de-hybridization and/or digestion of the template nucleic acid and permeabilization of the biological sample.

FIGS. 15A-15C is a schematic diagram showing a cycle of (15A) capture of a ligation product by hybridization of the ligation product with capture probes, (15B) extension of the capture probes and the ligation product and release or de-hybridization of the ligation product, and (15C) the extended capture probes immobilized on a substrate after release or de-hybridization of the ligation product.

FIG. 16 is a schematic diagram of four different pairs of barcoded templated ligation probes, each targeting a different analyte, respectively; and the extended capture probes corresponding to the four pairs of barcoded templated ligation probes, each targeting a different analyte, immobilized on a substrate.

FIG. 17 is a schematic diagram showing a first cycle of decoding four barcoded extended capture probes by fluorescent microscopy.

FIG. 18 is a schematic diagram showing a second cycle of decoding four barcoded extended capture probes by fluorescent microscopy.

FIG. 19 is a schematic diagram showing a third cycle of decoding four barcoded extended capture probes by fluorescent microscopy.

FIG. 20 is a schematic diagram showing a fourth cycle of decoding four barcoded extended capture probes by fluorescent microscopy.

FIG. 21 is a schematic drawing showing a set of four decoding primers (right) having a degenerate 3′-terminal nucleotide for resolving four extended capture probes (left) using a common barcode sequence and 3′-terminal mismatch base-pairing.

FIG. 22 is a schematic diagram showing a cycle of decoding four barcoded extended capture probes by fluorescent microscopy using a set of four decoding primers having a degenerate 3′-terminal nucleotide and a common barcode sequence.

FIG. 23 is a schematic diagram showing a cycle of decoding barcoded extended capture probes by successive rounds of hybridizing conjugated oligonucleotides and single-channel 2-bit imaging by microscopy.

### DETAILED DESCRIPTION

Targeted RNA capture is an attractive alternative to poly(A) mRNA capture for interrogating spatial gene expression in a sample (e.g., an FFPE tissue). Compared to poly(A) mRNA capture, targeted RNA capture as described herein is less affected by RNA degradation associated with FFPE fixation compared to methods dependent on capture of polyadenylated mRNA and reverse transcription of mRNA. Further targeted RNA capture as described herein allows for sensitive measurement of specific genes of interest that otherwise might be missed with a whole transcriptomic approach. Targeted RNA capture can be used to capture a defined set of RNA molecules of interest, or it can be used at a whole transcriptome level, or anything in between.

After RNA-templated ligation (RTL), also called templated ligation throughout, capture of ligation products on the array, and extension of capture probes, several methods exist for detecting capture sequences. When using barcoded templated ligation probes, unique combinations of the one or more barcodes of the first probe and the one or more barcodes of the second probe can be determined, and can specifically identify the RNA analyte to which the first and second probe hybridized. Barcoded probes can be detected on the array by microscopy, for example, fluorescent microscopy, electron microscopy, interferometric cross-polarization microscopy.

The compositions and methods described herein can be used to detect and decode signals from captured barcoded templated ligation probes either spatially in the context of a biological sample or after recovery from a library preparation. If a single channel of detection is used to detect unique combinations of barcodes, combinatorial 2-bit signatures identifying target analytes can read out using many cycles of annealing and combinatorial detection. Imaging one channel, for example by microscopy, is advantageously fast, and the signal for one-channel detection of beads or nanoparticles is advantageous in one plane on the array. This method of one-channel 2-bit detection also advantageously has very low background signal. Further, one-channel detection of hybridized oligonucleotide conjugates does not require amplification, saving time and reagent costs. Templated ligation probes can be designed to contain several barcodes, and barcodes may be multiplexed by, for example, using degenerate nucleotides at 3′-terminal end of decoding primers. The compositions and methods described herein can be used to detect and decode signals from captured barcoded templated ligation probes targeting a subset of transcripts in a biological sample, or targeting the entire transcriptome.

### I. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363 (6434): 1463-1467, 2019; Lee et al., Nat. Protoc. 10 (3): 442-458, 2015; Trejo et al., PLOS ONE 14 (2): e0212031, 2019; Chen et al., Science 348 (6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I) (b) of PCT Publication No. WO2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue. In some embodiments, the tissue sample is a solid tissue sample. In some embodiments, the biological sample is a tissue section. In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed using cryosectioning. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, the biological sample is not fixed with paraformaldehyde (PFA). In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, it is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, and paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9 (10): 5188-96; Kap M. et al., PLOS One; 6 (11): e27704 (2011); and Mathieson W. et al., Am J Clin Pathol.; 146 (1): 25-40 (2016), each of which are hereby incorporated by reference in their entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Non-limiting examples of stains include histological stains (e.g., hematoxylin and/or eosin) and immunological stains (e.g., fluorescent stains). The biological sample can be stained using Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. In some instances, PAS staining is performed after formalin or acetone fixation. In some embodiments, a biological sample (e.g., a fixed and/or stained biological sample) can be imaged. Biological samples are also described in Section (I)(d) of PCT Publication No. WO2020/176788 and/or U.S. Patent Application Publication No. 2020/0277663.

The following embodiments can be used with any of the methods described herein. In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding the primer to the biological sample.

In some embodiments, the method includes staining the biological sample. In some embodiments, the staining includes the use of hematoxylin and eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E),

Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). In some embodiments, a capture probe comprises a capture domain and one or more of a spatial barcode, a UMI and a cleavage domain. See, e.g., Section (II)(b)(e.g., subsections (i)-(vi)) of PCT Publication No. WO2020/176788 and/or U.S. Patent Application Publication No. 2020/0277663. Generation of capture probes can be achieved by any appropriate method, including those described in Section (II) (d) (ii) of PCT Publication No. WO2020/176788 and/or U.S. Patent Application Publication No. 2020/0277663

In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

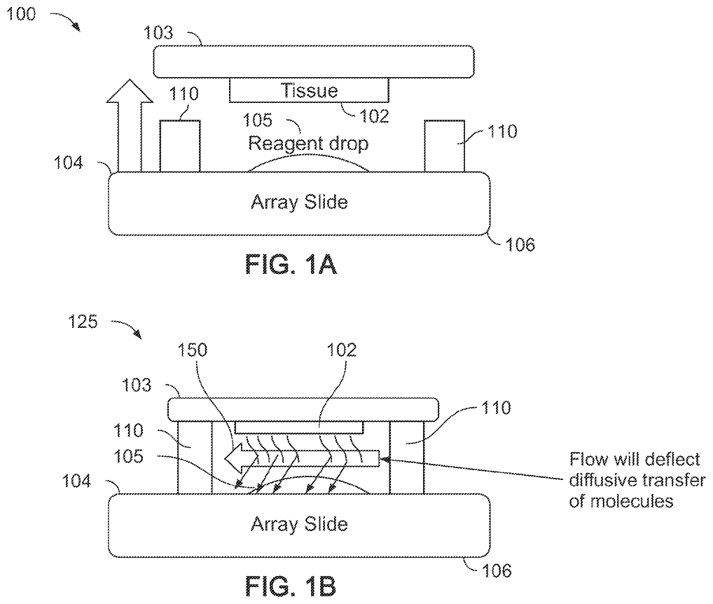

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A a liquid reagent drop 105 (e.g., comprising a permeabilization solution) is introduced on the second substrate in proximity to the capture probes 106 and in between the biological sample 102 and the second substrate (e.g., slide 104 including an array having spatially barcoded capture probes 106). The reagent drop 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent drop 105 within a gap between the first substrate (e.g., slide 103) and the second substrate (e.g., slide 104) creates a liquid interface between the two substrates. The reagent drop may comprise a permeabilization solution which permeabilizes and/or digests the biological sample 102. In some embodiments wherein the biological sample 102 has been pre-permeabilized, the reagent medium is not a permeabilization solution. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the reagent drop 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

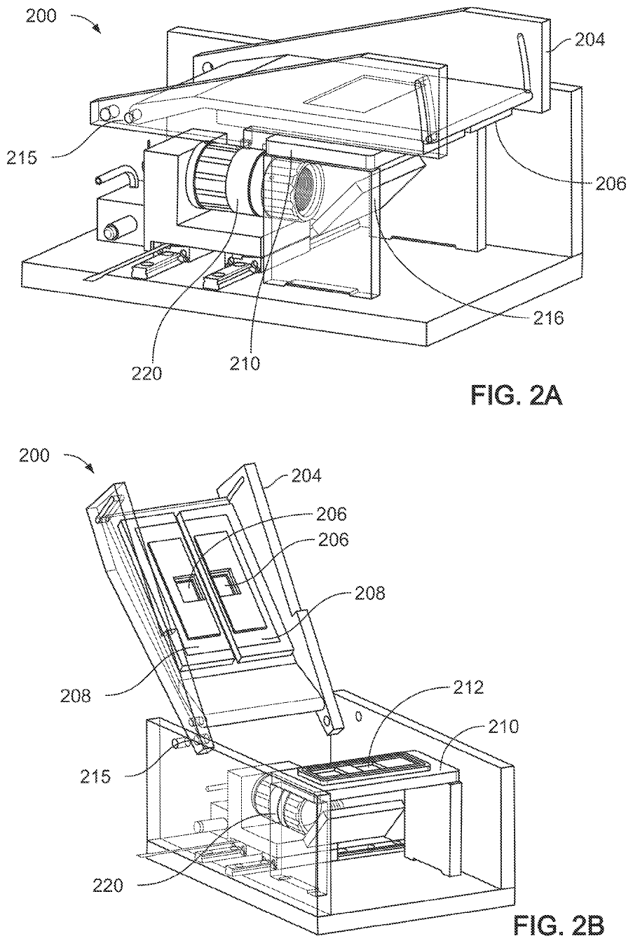

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

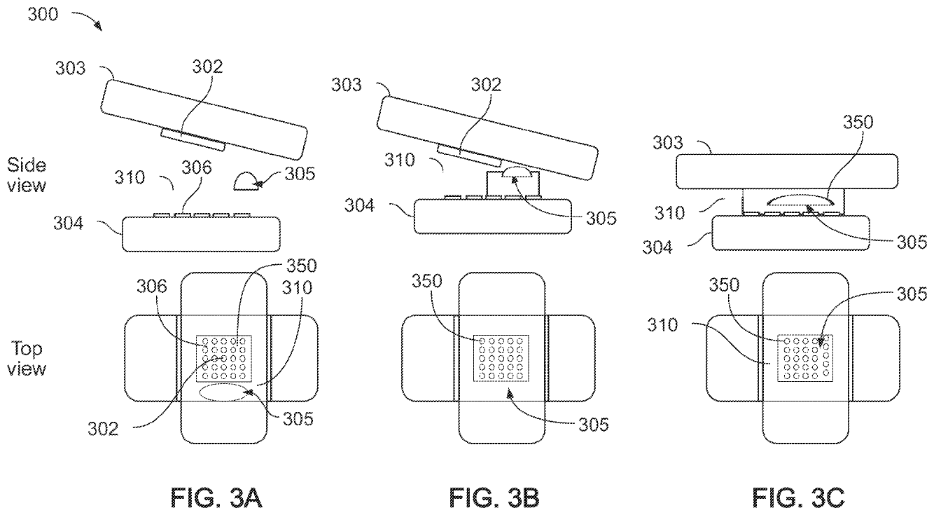

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step. In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

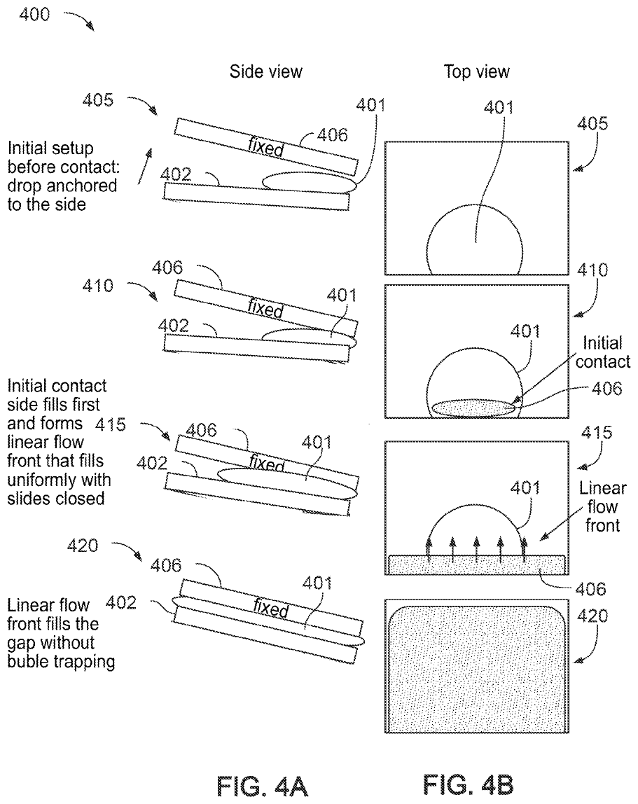

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates.

At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some embodiments, the reagent drop or medium (e.g., 105 in FIG. 1A) comprises a permeabilization agent. In some embodiments, following initial contact between the biological sample and a permeabilization agent, the permeabilization agent can be removed from contact with the biological sample (e.g., by opening sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, pepsin, elastase, and proteinase K. In some embodiments, the reagent medium comprises a nuclease. In some embodiments, the nuclease comprises an RNase. In some embodiments, the Rnase is selected from Rnase A, Rnase C, Rnase H, and Rnase I. In some embodiments, the reagent medium comprises one or more of sodium dodecyl sulfate (SDS) or a sodium salt thereof, proteinase K, pepsin, N-lauroylsarcosine, and RNAse.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In certain embodiments a dried permeabilization reagent is applied or formed as a layer on the first substrate or the second substrate or both prior to contacting the biological sample and the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for the template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended by a reverse transcriptase. In some embodiments, the capture probe is extended using one or more DNA polymerases. In some embodiments, the extended capture probes include the sequence of the capture domain and the sequence of the spatial barcode of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II) (a) of PCT Publication No. WO2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of PCT Publication No. WO2020/176788 and/or U.S. Patent Application Publication No. 2020/0277663. Some quality control measures are described in Section (II) (h) of PCT Publication No. WO2020/176788 and/or U.S. Patent Application Publication No. 2020/0277663.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II) (c) of PCT Publication No. WO2020/176788 and/or U.S. Patent Application Publication No. 2020/0277663. Exemplary features and geometric attributes of an array can be found in Sections (II) (d) (i), (II) (d) (iii), and (II) (d) (iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (e) of PCT Publication No. WO2020/176788 and/or U.S. Patent Application Publication No. 2020/0277663.

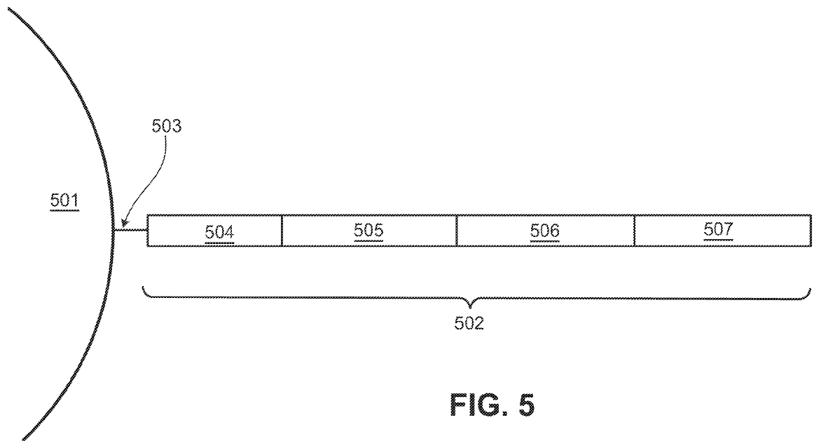

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that are useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte.

The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

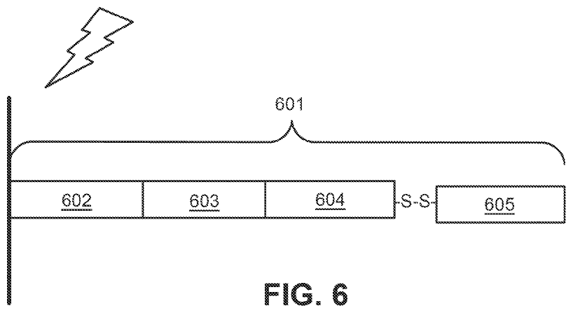

FIG. 6 is a schematic illustrating a cleavable capture probe, wherein the cleaved capture probe can enter into a non-permeabilized cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

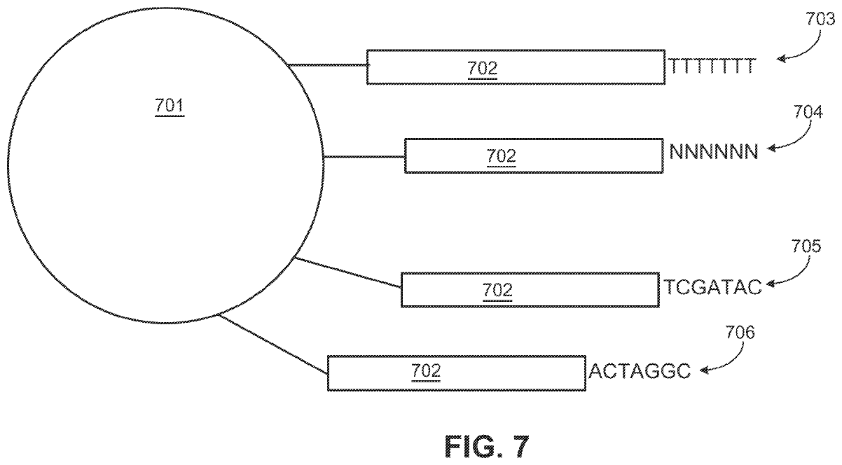

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 is common to all of the probes attached to a given feature. In some embodiments, the UMI sequence 506 of a capture probe attached to a given feature is different from the UMI sequence of a different capture probe attached to the given feature.

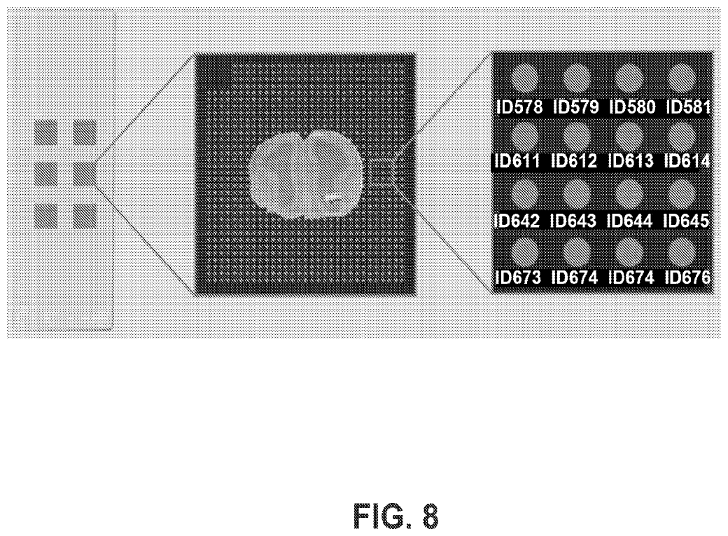

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture binding capture domain (e.g., a poly(A) sequence, a non-homopolymeric sequence). After hybridization to the analyte, a ligase (e.g., a T4 RNA ligase (Rnl2), a PBCV-1 DNA Ligase or Chorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

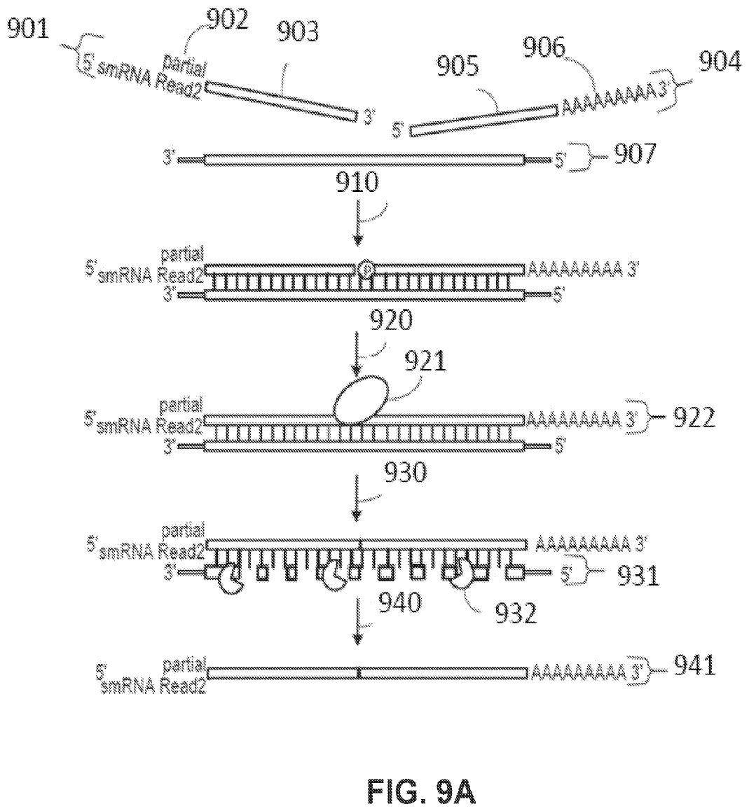

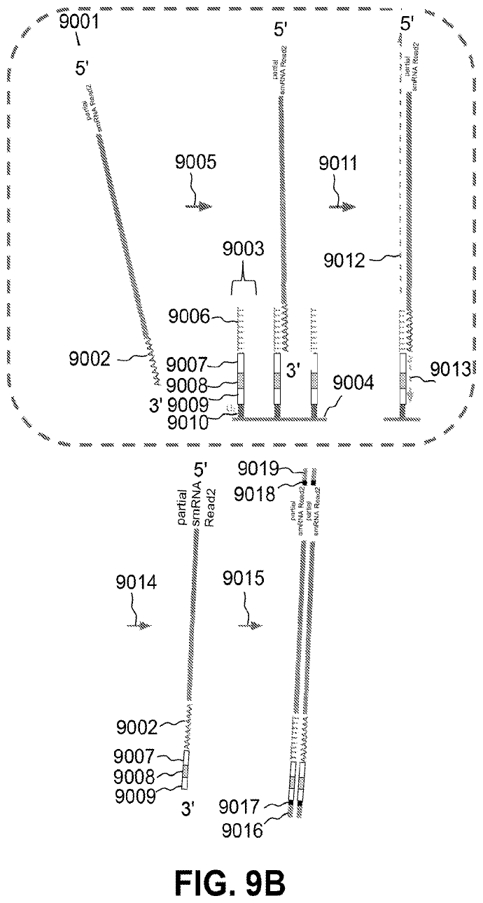

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily bind to the captured ligated probe (i.e., compared to no permeabilization). In some embodiments, reverse transcription (RT) reagents can be added to permeabilized biological samples. Incubation with the RT reagents can extend the capture probes 9011 to produce spatially-barcoded full-length cDNA 9012 and 9013 from the captured analytes (e.g., polyadenylated mRNA). Second strand reagents (e.g., second strand primers, enzymes) can be added to the biological sample on the slide to initiate second strand synthesis.

In some embodiments, cDNA can be denatured 9014 from the capture probe template and transferred (e.g., to a clean tube) for amplification, and/or library construction. The spatially-barcoded, full-length cDNA can be amplified 9015 via PCR prior to library construction. The cDNA can then be enzymatically fragmented and size-selected in order to optimize the cDNA amplicon size. P5 9016, i5 9017, i7 9018, and P7 9019, and can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663.

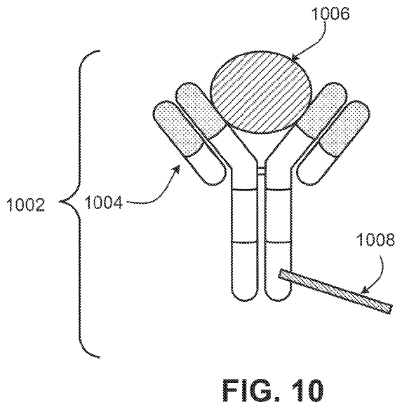

FIG. 10 is a schematic diagram of an exemplary analyte capture agent 1002 comprised of an analyte-binding moiety 1004 and an analyte-binding moiety barcode domain 1008. The exemplary analyte-binding moiety 1004 is a molecule capable of binding to an analyte 1006 and the analyte capture agent is capable of interacting with a spatially-barcoded capture probe. The analyte-binding moiety can bind to the analyte 1006 with high affinity and/or with high specificity. The analyte capture agent can include an analyte-binding moiety barcode domain 1008, a nucleotide sequence (e.g., an oligonucleotide), which can hybridize to at least a portion or an entirety of a capture domain of a capture probe. The analyte-binding moiety barcode domain 1008 can comprise an analyte binding moiety barcode and a capture handle sequence described herein. The analyte-binding moiety 1004 can include a polypeptide and/or an aptamer. The analyte-binding moiety 1004 can include an antibody or antibody fragment (e.g., an antigen-binding fragment).

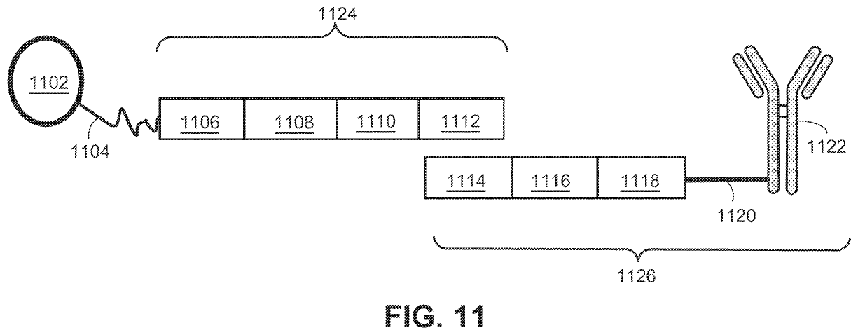

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and UMI 1110, as described elsewhere herein. The capture probe can be affixed 1104 to a feature (e.g., bead) or array 1102. The capture probe can also include a capture domain 1112 that is capable of binding to an analyte capture agent 1126. The analyte capture agent 1126 can include a functional sequence 1118, analyte binding moiety barcode 1116, and a capture handle sequence 1114 that is capable of binding to the capture domain 1112 of the capture probe 1124. The analyte capture agent can also include a linker 1120 that allows the capture agent barcode domain 1116 to couple to the analyte binding moiety 1122.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits-Tissue Optimization User Guide (e.g., Rev E, dated February 2022).

In some embodiments, spatial analysis can be performed using dedicated hardware and/or software, such as any of the systems described in Sections (II) (e) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Detecting Captured Ligation Products

The present disclosure utilizes both in situ and off-slide methods to detect analytes or derivatives of analytes. The methods utilize templated ligation and capture followed by detection using microscopy. This section describes methods of detection after a ligation product (e.g., from templated ligation methods described in Section (III) below) has been generated. Thus, after a ligation product from the sample has hybridized or otherwise been associated with a capture probe according to any of the methods described above in connection with the general spatial cell-based analytical methodology, the barcoded constructs that result from hybridization or association are analyzed. It is appreciated that the methods disclosed herein can be performed on a single slide (e.g., placing a biological sample onto a slide having spatial capture probes) or in a multiple slide (e.g., sandwiching) setup as shown in FIGS. 1-4B.

In some embodiments, provided herein are methods for spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte.

In certain embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest.

In some embodiments, the methods disclosed herein include providing the biological sample on an array comprising a plurality of capture probes, wherein a capture probe of the plurality of capture probes comprises a capture domain; contacting a first probe and a second probe with the biological sample, wherein the first probe and the second probe each comprise sequences that are substantially complementary to sequences of the nucleic acid, wherein the second probe comprises a capture probe capture domain that is complementary to all or a portion of the capture domain, wherein the first probe and/or the second probe comprises one or more barcode sequences selected from a plurality of barcode sequences, such that each combination of the first probe and the second probe comprises a unique combination of the one or more barcode sequences; hybridizing the first probe and the second probe to the nucleic acid; generating a ligation product by ligating the first probe and the second probe; releasing the ligation product from the nucleic acid; hybridizing the ligation product to the capture domain; and decoding the one or more barcode sequences or a complement thereof, and using the decoded one or more barcode sequences to determine the presence and/or location of the nucleic acid in the biological sample.

In some embodiments, the methods described herein can be of use in personalized medicine endeavors. In some embodiments, the methods disclosed herein can be of use in diagnostic assays. In some embodiments, the methods disclosed herein can be of use in determining or tracking treatment regimens for a subject with a disease or cancer. For example, a subject may be suspected of having a cancer or a disease state and the subject would provide a sample, such as a tissue sample, which can be permeabilized and identified target analyte(s) can hybridize to target specific probes comprising multiple barcodes as described herein, the probes can be ligated together and captured by complementary capture probe domains on a spatial array, and by decoding the multiple barcode sequences the presence of the targeted analyte(s) can be determined. The target analytes can be, for example, indicative of the presence of a cancer or disease, a stage or state of cancer or disease, or indicative of an expression pattern associated with a cancer or disease such as upregulation and/or downregulation of gene expression pattern. The multiple barcodes are used to identify the gene expression patterns. Additionally, the method previously described can be used similarly to track the success, or failure, of a treatment regimen in a subject diagnosed with a cancer or a disease. For example, by taking tissue samples from a subject at different time points in a treatment regimen, the methods described above can be used to follow any changes to the target analyte(s) and/or gene expression pattern in assessing the success or failure of a treatment. The tracking of success or failure of a treatment regimen can then allow a practitioner to adjust a treatment for a subject or to determine whether a change in treatment in the subject is necessary.

In some embodiments, the tissue includes is a tumor (e.g., a malignant or a benign tumor). In some embodiments, the tumor is a solid tumor. In some embodiments, the subject is suspected of having a cancer. In some embodiments, the subject has been previously diagnosed or identified as having a cancer (e.g., any of the exemplary cancers described herein).

Non-limiting examples of cancers referred to in any one the methods described herein include: sarcomas, carcinomas, adrenocortical carcinoma, AIDS-related cancers, anal cancer, appendix cancer, astrocytomas, atypical teratoid/rhabdoid tumor, basal cell carcinoma, bladder cancer, brain stem glioma, brain tumors (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors, and pineoblastoma), breast cancer, bronchial tumors, cancer of unknown primary site, carcinoid tumor, carcinoma of unknown primary site, central nervous system atypical teratoid/rhabdoid tumor, central nervous system embryonal tumors, cervical cancer, childhood cancers, chordoma, colon cancer, colorectal cancer, craniopharyngioma, endocrine pancreas islet cell tumors, endometrial cancer, ependymoblastoma, ependymoma, esophageal cancer, esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioma, head and neck cancer, heart cancer, hypopharyngeal cancer, intraocular melanoma, islet cell tumors, Kaposi's sarcoma, kidney cancer, Langerhans cell histiocytosis, laryngeal cancer, lip cancer, liver cancer, lung cancer, malignant fibrous histiocytoma bone cancer, medulloblastoma, medulloepithelioma, melanoma, Merkel cell carcinoma, Merkel cell skin carcinoma, mesothelioma, metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, non-melanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pineal parenchymal tumors of intermediate differentiation, pineoblastoma, pituitary tumor, pleuropulmonary blastoma, primary hepatocellular liver cancer, prostate cancer, rectal cancer, renal cancer, renal cell (kidney) cancer, renal cell cancer, respiratory tract cancer, retinoblastoma, rhabdomyosarcoma, salivary gland cancer, Sezary syndrome, small cell lung cancer, small intestine cancer, soft tissue sarcoma, squamous cell carcinoma, squamous neck cancer, stomach (gastric) cancer, supratentorial primitive neuroectodermal tumors, testicular cancer, throat cancer, thymic carcinoma, thymoma, thyroid cancer, transitional cell cancer, transitional cell cancer of the renal pelvis and ureter, trophoblastic tumor, ureter cancer, urethral cancer, uterine cancer, uterine sarcoma, vaginal cancer, vulvar cancer, Waldenström macroglobulinemia, and Wilm's tumor.

The decoding step as described herein includes methods of decoding an extended capture probe using fluorescent and particle detection. In some embodiments, a capture probe can be extended (an “extended capture probe,” e.g., as described herein). For example, extending a capture probe can include generating an extension product that is complementary to a captured (hybridized) ligation product. This process involves synthesis of a complementary strand of the hybridized nucleic acid, e.g., generating an extended capture probe based on the captured ligation product (the template ligation product hybridized to the capture domain of the capture probe). Thus, in an initial step of extending a capture probe, e.g., the extension product generation, the captured (hybridized) nucleic acid, e.g., templated ligation product, acts as a template for the extension step.

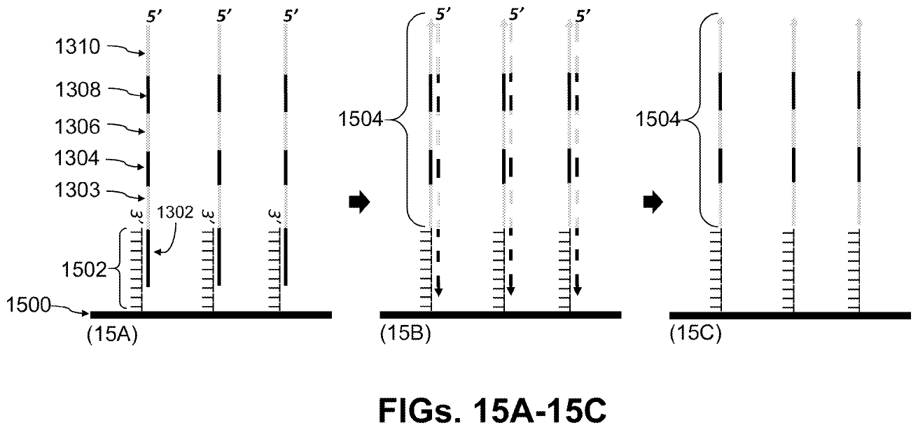

As shown in FIGS. 15A-15C, a non-limiting example of a ligation product including a first probe ligated to a second probe, hybridized to a capture probe 1502 affixed to a substrate 1500, wherein the first probe comprises a first barcode sequence 1310, a second barcode sequence 1308, and a sequence 1306 that is substantially complementary to a first target sequence in the analyte, and the second probe comprises a sequence 1304 that is substantially complementary to a second target sequence in the analyte, a third barcode sequence 1303, and a capture probe capture domain 1302 that is hybridized to capture probe 1502. Barcode sequences 1310, 1308, and 1303 are selected from a plurality of unique barcode sequences, such that the combination of barcode sequences 1310, 1308, and 1303 specifically identifies the analyte targeted by the first and second probes. Capture probe 1502 is extended using the captured ligation product as a template to produce extended capture probe 1504. The ligation product is cleaved or de-hybridized, leaving extended capture probe 1504 immobilized on substrate 1500. Extended capture probe 1504 includes polynucleotide sequences, or reverse complements thereof, corresponding to the first barcode sequence 1310, the second barcode sequence 1308, and the third barcode sequence 1303, such that the unique combination of the barcode sequences specifically identifies the RNA analyte to which the first and second probes hybridized.

In some embodiments, the capture probe is extended using reverse transcription. For example, reverse transcription includes synthesizing cDNA from the ligation product, using a reverse transcriptase. In some embodiments, reverse transcription is performed while the tissue is still in place (or alternatively off the slide), generating a nucleic acid library, where the nucleic acid library includes the spatial barcodes from the adjacent capture probes. In some embodiments, the capture probe is extended using one or more DNA polymerases. In some embodiments, the tissue is removed prior to generating a nucleic acid library.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule or ligation product. In some embodiments, 3′ end of the extended probes, e.g., first strand cDNA molecules, is modified. For example, a linker or adaptor can be ligated to the 3′ end of the extended probes. This can be achieved using single stranded ligation enzymes such as T4 DNA ligase (available from Lucigen, Middleton, WI).

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended capture probe.

In some embodiments, the probes can be complementary to a single nucleic acid sequence. In some embodiments, the probes can be complementary to one or more nucleic acid sequences. In some embodiments, a plurality of probes, each conjugated to a different detection moiety, can be complementary to a single nucleic acid sequence, for example, a barcode sequence within an extended capture probe. In some embodiments, the probes (e.g., detectable probes) can be directed to a panel of nucleic acids associated with a disease (e.g., cancer, Alzheimer's disease, Parkinson's disease).

### A. Detecting Captured Probes by Fluorescent Microscopy

After extension of the capture probe to produce an extended capture probe, some or all of the sequence or identity of the extended capture probe can be determined. In some embodiments, the sequence identities the one or more barcodes of the first probe and the one or more barcodes of the second probe are determined. In some embodiments the unique combination of the one or more barcodes of the first probe and the one or more barcodes of the second probe are determined, and can thereby identify the RNA sequence to which the first and second probes hybridized. In some embodiments, the spatial location of the RNA, as an example, within the biological sample can be determined based on unique combination of the one or more barcodes of the first probe and the one or more barcodes of the second probe. Probes can be detected on the array by, for example, microscopy. In some embodiments, probes are detected on the array by fluorescent microscopy, as discussed in further detail below.

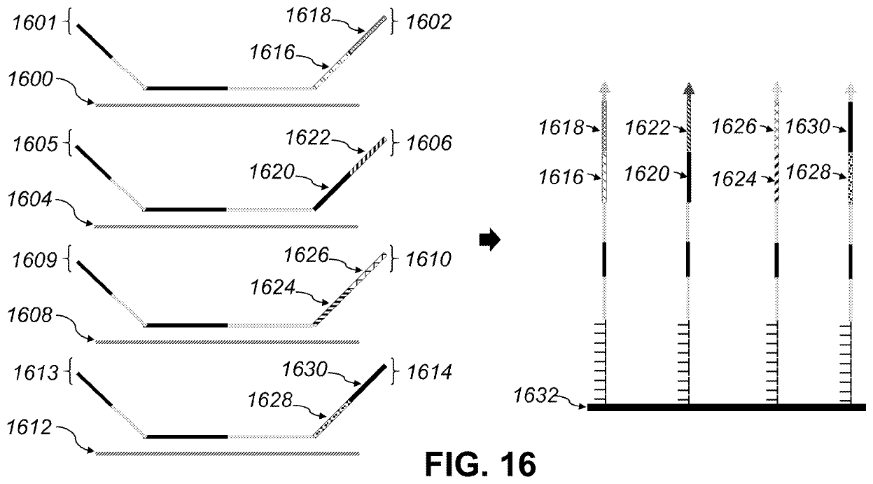

As shown in FIGS. 16-20, a non-limiting example of detection of unique combinations of barcodes within extended capture probes is provided. As discussed above, pairs of probes may be designed to target specific nucleic acid sequences within a biological sample, e.g., DNA probes may be designed to target mRNA analytes. Referring to FIG. 16, probes 1601 and 1602 target mRNA 1600; probes 1605 and 1606 target mRNA 1604; probes 1609 and 1610 target mRNA 1608; probes 1613 and 1614 target mRNA 1612. Probe 1602 comprises unique barcode sequences 1616 and 1618, which are specifically associated with target mRNA 1600; Probe 1606 comprises unique barcode sequences 1620 and 1622, which are specifically associated with target mRNA 1604; Probe 1610 comprises unique barcode sequences 1624 and 1626, which are specifically associated with target mRNA 1608; Probe 1614 comprises unique barcode sequences 1628 and 1630, which are specifically associated with target mRNA 1612. After hybridization of the first and second probes to the target mRNA, ligation to produce a ligation product, release of the analyte, hybridization of the ligation product to the capture domain of the capture probe, and extension to produce an extended capture probe, a plurality of extended capture probes are immobilized on substrate 1632, with each extended capture probe comprising a unique combination of barcode sequences specifically identifying the target mRNA to which the first and second probes hybridized to within the biological sample.

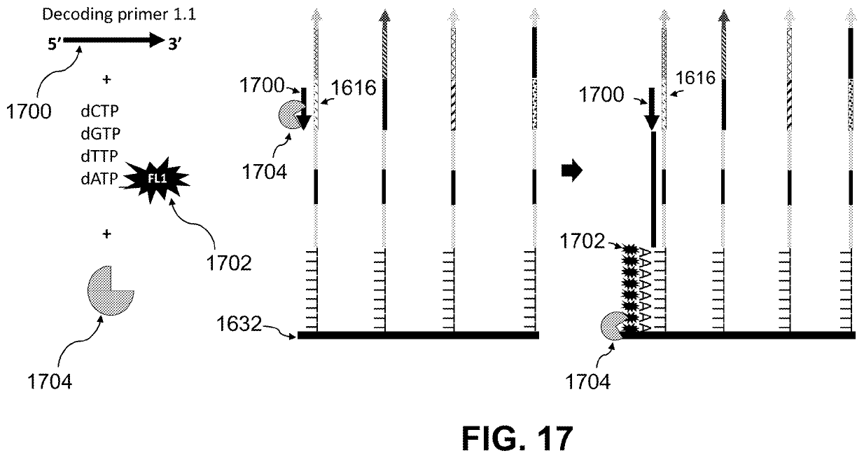

Referring to FIG. 17, extended capture probes may be detected on substrate 1632 by one or more successive rounds of hybridization and detection of a “decoding primer” to unique barcode sequences within the extended capture probes and polymerization using modified nucleotides (e.g., fluorescently labelled nucleotides) and using the extended capture probe as a template. In some embodiments, a decoding primer 1700 is added to the extended capture probes on the array, where the decoding primer hybridizes to one of the plurality of unique barcode sequences of the first or the second probe, for example, decoding primer 1700 specifically hybridizes to barcode sequence 1616. A mixture of dNTPs (dATP, dTTP, dCTP, and dGTP) and a polymerase 1704 are added to the array, wherein the dATP species are labeled with a first fluorophore 1702. The decoding primer 1700 is extended by polymerase 1704 and the product is labeled by incorporation of the labelled dATP 1702 during polymerization. In some embodiments, the capture probe capture domain of the first probe comprises a poly(A) sequence, such that a string of 1, 2, 3, 4, 5, 6, 7, 8, 9, 10, 11, 12, 13, 14, 15, 16, 17, 18, 19, 20, 30, 40, 50 or more fluorophore 1702 species are incorporated into the extension product initiated from the decoding primer 1700. After the decoding primer 1700 is extended and the modified nucleotides are incorporated into the extension product, excess primers, polymerase, and dNTPs are washed from the array.

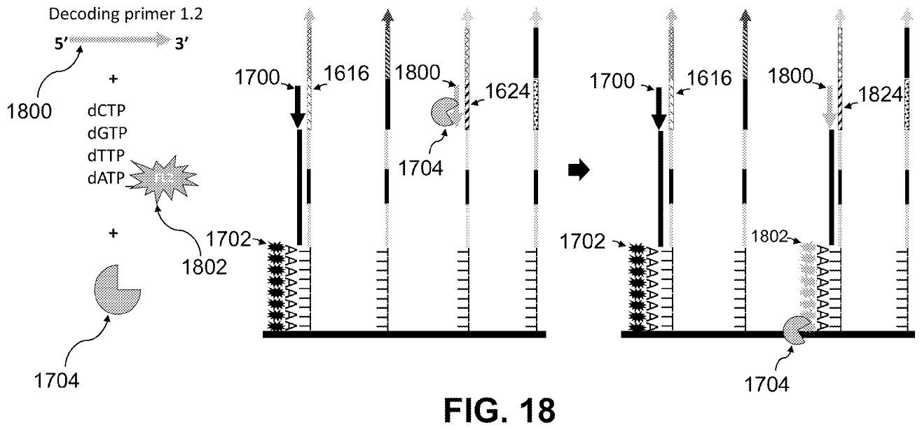

Referring to FIG. 18, a second round of hybridization of a second “decoding primer” to a different unique barcode sequence is shown. A second decoding primer 1800 is added to the array, where the decoding primer hybridizes to one of the plurality of unique barcode sequences of the first or the second probe on the extended capture probe, for example, decoding primer 1800 specifically hybridizes to barcode sequence 1624. A mixture of dNTPs (dATP, dTTP, dCTP, and dGTP) and a polymerase 1704 are added to the array, wherein the dATP species are labeled with a second fluorophore 1802. The second decoding primer 1800 is extended by polymerase 1704 and the product is labeled by the second fluorophore 1802. In some embodiments, the capture probe capture domain of the first probe comprises a poly(A) sequence, such that a string of 1, 2, 3, 4, 5, 6, 7, 8, 9, 10, 11, 12, 13, 14, 15, 16, 17, 18, 19, 20, 30, 40, 50 or more fluorophore 1802 species are incorporated into the extension product initiated from the decoding primer 1800. After the decoding primer 1800 is extended and the modified nucleotides are incorporated into the extension product, excess primers, polymerase, and dNTPs are washed from the array.

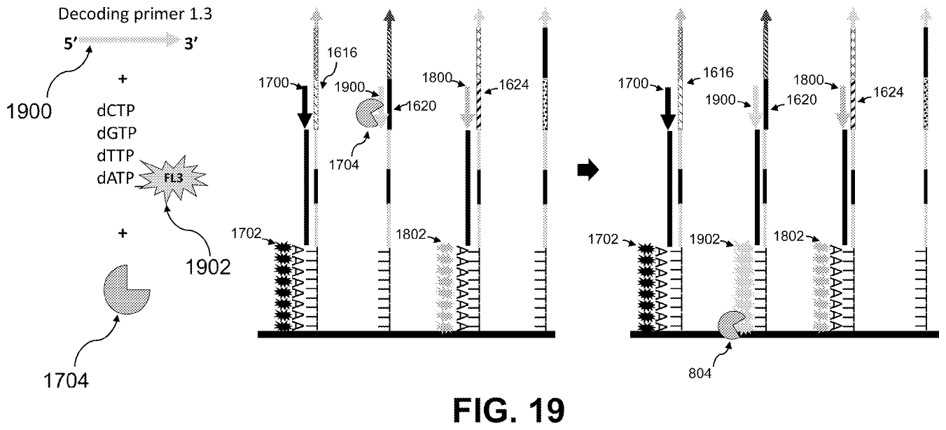

Referring to FIG. 19, a third round of hybridization of a third “decoding primer” to a different unique barcode sequence is shown. A third decoding primer 1900 is added to the array, where the decoding primer hybridizes to one of the plurality of unique barcode sequences of the first or the second probe of the extension product, for example, decoding primer 1900 specifically hybridizes to barcode sequence 1620. A mixture of dNTPs (dATP, dTTP, dCTP, and dGTP) and a polymerase 1704 are added to the array, wherein the dATP species are labeled with a third fluorophore 1902. The third decoding primer 1900 is extended by polymerase 1704 and the product is labeled by the third fluorophore 1902. In some embodiments, the capture probe capture domain of the first probe comprises a poly(A) sequence, such that a string of 1, 2, 3, 4, 5, 6, 7, 8, 9, 10, 11, 12, 13, 14, 15, 16, 17, 18, 19, 20, 30, 40, 50 or more fluorophore 1902 species are incorporated into the extension product initiated from the decoding primer 1900. After the decoding primer 1900 is extended and the modified nucleotides are incorporated into the extension product, excess primers, polymerase, and dNTPs are washed from the array.

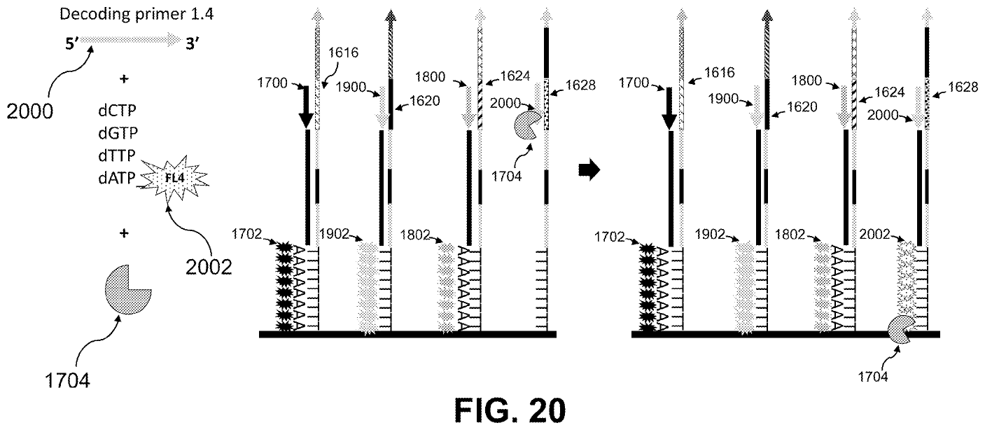

Referring to FIG. 20, a fourth round of hybridization of a fourth “decoding primer” to a different unique barcode sequence is shown. A fourth decoding primer 2000 is added to the array, where the decoding primer hybridizes to one of the plurality of unique barcode sequences of the first or the second probe of the extension product, for example, decoding primer 2000 specifically hybridizes to barcode sequence 1628. A mixture of dNTPs (dATP, dTTP, dCTP, and dGTP) and a polymerase 1704 are added to the array, wherein the dATP species are labeled with a fourth fluorophore 2002. The fourth decoding primer 2000 is extended by polymerase 1704 and the product is labeled by the fourth fluorophore 2002. In some embodiments, the capture probe capture domain of the first probe comprises a poly(A) sequence, such that a string of 1, 2, 3, 4, 5, 6, 7, 8, 9, 10, 11, 12, 13, 14, 15, 16, 17, 18, 19, 20, 30, 40, 50 or more fluorophore 2002 species are incorporated into the extension product initiated from the decoding primer 2000. After the decoding primer 2000 is extended and the modified nucleotides are incorporated into the extension product, excess primers, polymerase, and dNTPs are washed from the array.

As such, the decoding can be of a cyclic nature using multiple fluorescently labeled nucleotides in subsequent cycles to determine the presence of the multiple barcodes present.

In some embodiments, after four cycles of extension, with the extended capture probes being decorated with four different species of fluorophore, the array is washed and imaged by fluorescence microscopy. In some embodiments, 1, 2, 3, 4, 5, 6, or more cycles of extension with 1, 2, 3, 4, 5, 6, or more different species of fluorophore may be performed. In some embodiments, further rounds of hybridization of a decoding primer, extension of the decoding primer with modified nucleotides comprising fluorescently labeled dATPs, and imaging by fluorescent microscopy, are performed to further resolve the spatial distribution of captured probes on the array. For example, referring to FIG. 16, a first set of rounds of extension and detection may be directed to barcode sequences 1616, 1620, 1624, and 1628 as described above, and a second set of rounds of extension and detection may be directed to barcode sequences 1618, 1622, 1626, and 1630. After each set of four cycles of extension, with the extended capture probes being decorated with four different species of fluorophore, the array is washed and imaged by high resolution fluorescence microscopy. The resulting high-resolution fluorescent images may be analyzed computationally to spatially map the analyte within the biological sample on the array, with detection of unique combinations of barcode sequences specifically identifying analytes within the biological sample.

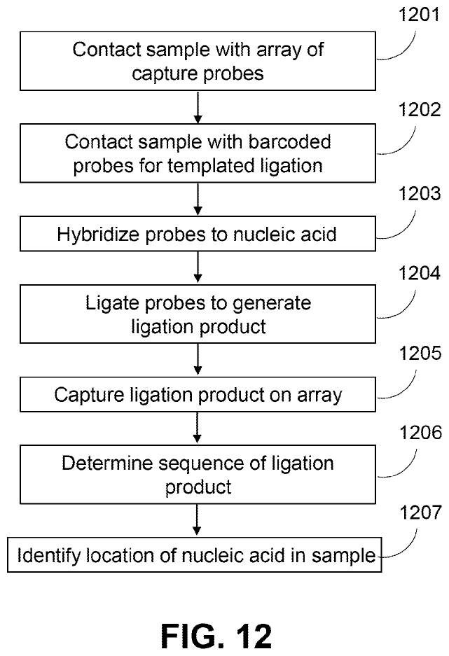

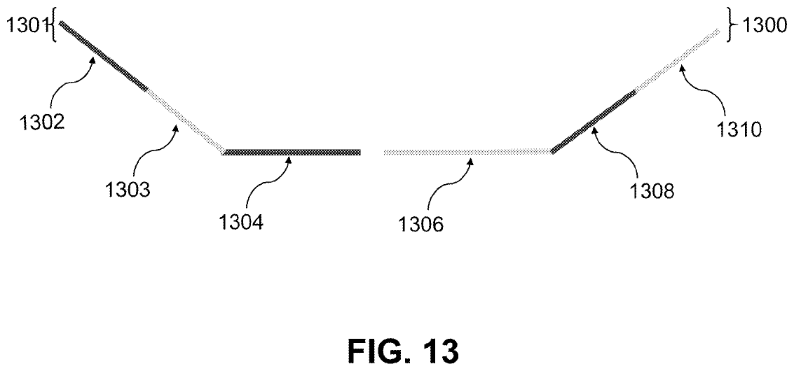

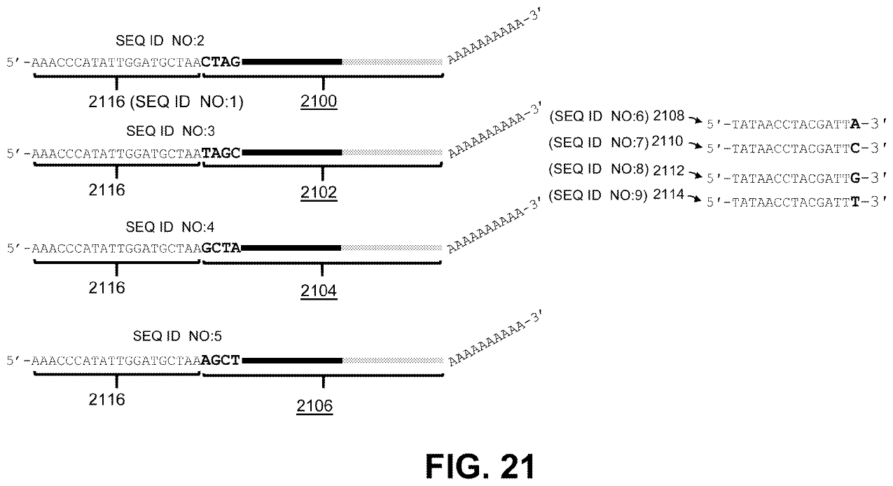

In some embodiments, to increase multiplex capabilities and reduce the length of the barcodes within the first and the second probes, a decoding primer that hybridizes to a common barcode sequence assigned to multiple target analytes may be used, where 3′-terminal nucleotide of the decoding primer differentiates between multiple analyte target sequences. It will be appreciated by a person of ordinary skill in the art that a 3′-terminal nucleotide mismatch dramatically reduces the efficiency of a primer annealing to its target, and 3′-terminal nucleotide mismatches may be used to specifically amplify a particular template. As shown in FIGS. 12-13, a non-limiting example of detection of unique combinations of barcodes using a decoding primer that hybridizes to a common barcode sequence assigned to multiple target nucleic acids is shown. Ligation product sequences 1200, 1202, 1204, and 1206 correspond to four different target nucleic acid sequences within a biological sample. In some embodiments, a single barcode sequence is included in the probe sequence associated with 1, 2, 3, or 4 extended capture probe sequence, and decoding primers are designed such that the primer hybridizes to the common barcode sequence but the 3′-terminal nucleotide of the primer is degenerate, resulting in only one decoding primer of four possible degenerate primers amplifying the extended capture probe sequence. Referring to FIG. 21, decoding primers 2108, 2110, 2112, and 2114 each hybridize to barcode sequence 2116, however, because of the degenerate 3′-terminal nucleotide of decoding primers 2108, 211110, 2112, and 2114, decoding primer 2108 specifically hybridizes to ligation product sequence 2102, decoding primer 2110 specifically hybridizes to ligation product sequence 2104, decoding primer 2112 specifically hybridizes to ligation product sequence 2100, and decoding primer 2114 specifically hybridizes to ligation product sequence 2106.

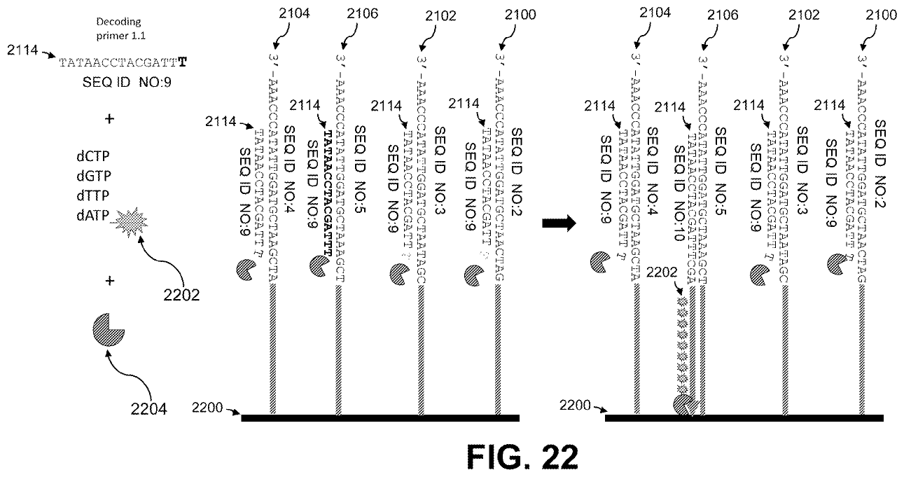

Referring to FIG. 22, similar to the decoding method depicted in FIGS. 16-20, extended capture probes can be detected on substrate 2200 by one or more successive rounds of hybridization of a decoding primer to a common barcode sequence within the extended capture probe and polymerization using modified nucleotides, with a 3′-terminal nucleotide of the decoding primer distinguishing between one, two, three, or four extended capture probe sequences for selective amplification of one extended capture probe sequence. A decoding primer 2114 is added to the extended capture probes on the array, where the decoding primer hybridizes to one of the plurality of common barcode sequences of the first or the second probe. Due to its 3′-terminal nucleotide, decoding primer 2114 specifically hybridizes to and extends ligation product sequence 2106 and does not extend ligation product sequences 2100, 2102, and 2104. A mixture of dNTPs (dATP, dTTP, dCTP, and dGTP) and a polymerase 2204 are added to the array, wherein the dATP species are labeled with a fluorophore 2202. The decoding primer 2114 is extended by polymerase 2204 and the product is labeled by the fluorophore 2202. In some embodiments, the capture probe capture domain of the first probe comprises a poly(A) sequence, such that a string of 1, 2, 3, 4, 5, 6, 7, 8, 9, 10, 11, 12, 13, 14, 15, 16, 17, 18, 19, 20, 30, 40, 50 or more fluorophore 2202 species are incorporated into the extension product initiated by the decoding primer 2114. After the decoding primer 2114 is extended and the modified nucleotides are incorporated into the extension product, excess primers, polymerase, and dNTPs are washed from the array. Utilizing 3′-terminal nucleotide specificity of the decoding primers allows for up to four extended capture probe species to be resolved by a set of four primers that hybridize to a single barcode sequence.

In some embodiments, after four cycles of extension utilizing a set of four primers targeted to a single barcode sequence with degenerate 3′-terminal nucleotide differentiating four extended capture probe species, with the extended capture probes being decorated with four different species of fluorophore, the array is washed and imaged by high resolution fluorescence microscopy. In some embodiments, 1, 2, 3, 4, 5, 6, or more cycles of extension with 1, 2, 3, 4, 5, 6, or more different species of fluorophore may be performed. In some embodiments, further rounds of hybridization of a decoding primer, extension of the decoding primer with modified nucleotides comprising fluorescently labeled dATPs, and imaging by fluorescent microscopy, are performed to further resolve the spatial distribution of captured probes on the array.

### B. Detecting Captured Probes by Electron Microscopy

After extension of the capture probe to produce an extended capture probe, some or all of the sequence of the extended capture probe can be identified. In some embodiments, the sequence identities the one or more barcodes of the first probe and the one or more barcodes of the second probe are determined. In some embodiments the unique combination of the one or more barcodes of the first probe and the one or more barcodes of the second probe are determined, and can specifically identify the RNA analyte to which the first and second probe hybridized. In some embodiments the spatial location of a target nucleic acid within the biological sample can be determined based on unique combination of the one or more barcodes of the first probe and the one or more barcodes of the second probe. Probes can be detected on the array by, for example, fluorescent microscopy. In other embodiments, probes are detected on the array by electron microscopy. In other embodiments, probes are detected on the array by interferometric cross-polarization microscopy.

As described above, probes for templated ligation can be designed such that the first and the second probe each comprise one or more unique barcode sequences, and the ligation product of the combined first probe and second probe can comprise a unique combination of barcode sequences that identify the nucleic acid targeted by the probes. These unique combinations of barcodes allow for cycles of detection and combinatorial detection by hybridizing oligonucleotides conjugated to, for example, nanoparticles, spheres, semiconductor particles, quantum dots, or any conjugate that can be detected at single-molecule resolution. If a single channel of detection is used, barcodes of 2 bits can be created using many cycles of annealing and combinatorial detection. Imaging one channel, for example by microscopy, is advantageously fast, and the signal for one-channel detection of beads or nanoparticles is advantageous in one plane on the array. This method of one-channel 2-bit detection also advantageously has very low background signal. Further, one-channel detection of hybridized oligonucleotide conjugates does not require amplification, saving time and reagent costs.

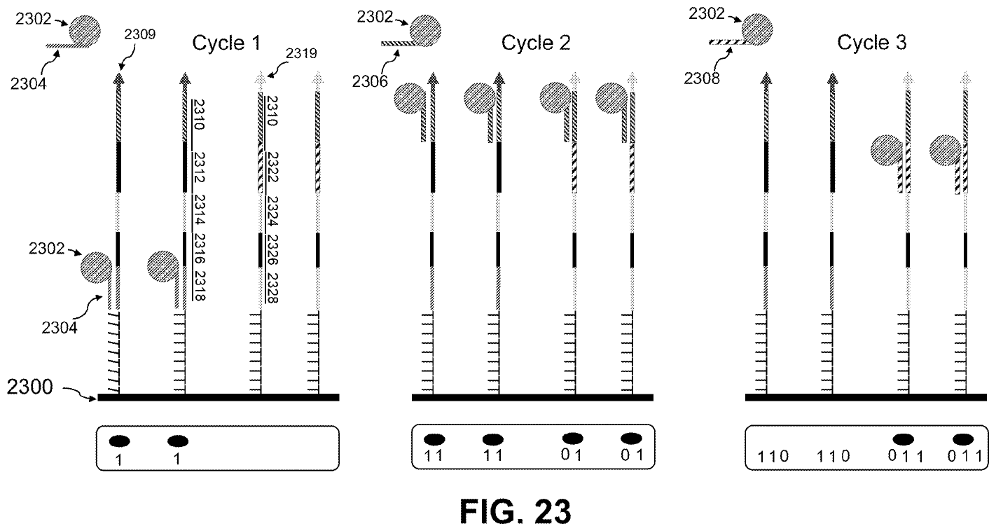

As shown in FIG. 23, a non-limiting example of detection of unique combinations of barcodes within extended capture probes by hybridizing oligonucleotides conjugated to nanoparticles is provided. Extended capture probe 2309 comprises a first barcode sequence 2310, a second barcode sequence 2312, sequence 2314 corresponding to the first target sequence in the nucleic acid, sequence 2316 corresponding to the second target sequence in the nucleic acid, a third barcode sequence 2318, and as depicted in FIG. 23 there are two copies of extended capture probe 2309 immobilized on substrate 2300. Extended capture probe 2319 represents a different target nucleic acid than extended capture probe 2309 and comprises the same first barcode sequence 2310, a second barcode sequence 2322, sequence 2324 corresponding to the first target sequence in the nucleic acid, sequence 2326 corresponding to the second target sequence in the nucleic acid, a third barcode sequence 2388, and as depicted in FIG. 23 there are two copies of extended capture probe 2319 immobilized on substrate 2300.

For a first cycle of 2-bit combinatorial barcode detection, oligonucleotide 2304 conjugated to nanoparticle 2302 is contacted to the array. Oligonucleotide 2304 is complementary to barcode sequence 2318, resulting in the nanoparticle conjugated oligonucleotides labeling the two copies of extended capture probe 2309. The array is imaged and signal from the nanoparticle 2302 is recorded and mapped at high resolution. Only subsets of target nucleic acid signals are recorded and decoded at every cycle. Oligonucleotides are de-hybridized and washed away to clear the array for the next cycle.

For a second cycle of 2-bit combinatorial barcode detection, oligonucleotide 2306 conjugated to nanoparticle 2302 is contacted to the array. Oligonucleotide 2306 is complementary to barcode sequence 2310, resulting in the nanoparticle conjugated oligonucleotides labeling the two copies of extended capture probe 2309 and the two copies of extended capture probe 2319. The array is imaged and signal from the nanoparticle 2302 is recorded and mapped at high resolution.

For a third cycle of 2-bit combinatorial barcode detection, oligonucleotide 2308 conjugated to nanoparticle 2302 is contacted to the array. Oligonucleotide 2308 is complementary to barcode sequence 2322, resulting in the nanoparticle conjugated oligonucleotides labeling the two copies of extended capture probe 2319 and not the two copies of extended capture probe 2309. The array is imaged and signal from the nanoparticle 2302 is recorded and mapped at high resolution.

Repeated cycles of hybridization and detection produce a 2-bit signature for each extended capture probe on the array that uniquely identifies and maps the target analyte corresponding to the probes.

In some embodiments, the extended capture probes are hybridized to oligonucleotides conjugated to nanoparticles. In some embodiments, the nanoparticles are about 1, 2, 3, 4, 5, 6, 7, 8, 9, or 10 nm in diameter. In some embodiments, the nanoparticles are metal nanoparticles. In some embodiments, the nanoparticles are gold nanoparticles. In some embodiments, the extended capture probes are hybridized to oligonucleotides conjugated to beads. In some embodiments, the beads are about 1, 2, 3, 4, 5, 6, 7, 8, 9, or 10 nm in diameter.

In some embodiments, conjugated oligonucleotides hybridized to extended capture probes are detected by microscopy. In some embodiments, conjugated oligonucleotides hybridized to extended capture probes are detected by low-energy scanning electron microscopy. In some embodiments, conjugated oligonucleotides hybridized to extended capture probes are detected by interferometric cross-polarization microscopy.

### III. Analyte Capture Using Templated Ligation

Although techniques such as whole genome sequencing and whole exome sequencing are available, these techniques have drawbacks in that they provide copious amounts of information and increase costs for an experiment. In situations where one prefers to examine a more limited number of analytes, methods herein are provided for targeted nucleic acid capture. Capturing a derivative of a target nucleic acid (e.g., a ligation product) provides enhanced specificity with respect to detection and identification of the target nucleic acid. This is because at least two probes specific for a target are required to hybridize to the target in order to facilitate ligation and ultimate capture of the target nucleic acid derivative or proxy. It is appreciated that the methods disclosed herein can be performed on a single slide (e.g., placing a biological sample onto a slide having capture probes) or in a multiple slide (e.g., sandwiching) setup as shown in FIGS. 1-4B.

Methods of templated ligation have been described previously in WO 2021/133849, US 2021/0348221, and US 2021/0285046, each of which is incorporated by reference in its entirety.

Referring to FIG. 12, in an exemplary embodiment of the disclosure, provided are methods for identifying a location of a target nucleic acid in a biological sample. In some instances, the methods include 1201 contacting a biological sample with array of capture probes. In some instances, the array is on a substrate and the array includes a plurality of capture probes, wherein a capture probe of the plurality includes a capture domain. In some instances, the capture domain includes a poly(T) sequence. After placing the biological sample on the array, the biological sample 1202 is contacted with a first probe and a second probe, wherein the first probe and the second probe each include one or more sequences that are substantially complementary to sequences of the target nucleic acid and one or more barcode sequences selected from a plurality of barcode sequences and wherein the second probe includes a capture probe capture domain; the first probe and the second probe 1203 hybridize to complementary sequences in the target nucleic acid. After hybridization a ligation product comprising the first probe and the second probe 1204 is generated, and the ligation product is released from the target nucleic acid. The liberated ligation product is then able 1205 to hybridize to the capture domain of a capture probe on the array. After capture, all or a part of the sequence of the ligation product specifically bound to the capture domain, or a complement thereof can be determined by one of several methods described in further detail below, and the determined sequence of 1207 can be used to identify the location of the target nucleic acid in the biological sample. Target nucleic acid capture using templated ligation is depicted in FIG. 9A.

Also provided herein are methods for identifying a location of a target nucleic acid in a biological sample that includes a second probe including a pre-adenylated phosphate group at its 5′ end, which enables the ligating to use a ligase that does not require adenosine triphosphate for ligase activity.

Also provided herein are methods for identifying a location of a target nucleic acid in a biological sample that includes optimized hybridizing, washing, and releasing steps, as described above and shown in FIG. 9B.

In some embodiments, methods provided herein include permeabilization of the biological sample such that the capture probe can more easily hybridize to the captured ligation product (i.e., compared to no permeabilization). In some embodiments, reverse transcription (RT) reagents can be added to permeabilized biological samples. The methods provided herein utilize probe pairs (or sets; the terms are interchangeable). In some instances, the probe pairs are designed so that each probe hybridizes to a sequence in a target nucleic acid that is specific to that target nucleic acid (e.g., compared to the entire genome or transcriptome). That is, in some instances, a single probe pair can be specific to a single target nucleic acid.

In other embodiments, probes can be designed so that one of the probes of a pair is a probe that hybridizes to a specific sequence. The other probe can be designed to detect a mutation of interest. Accordingly, in some instances, multiple second probes can be designed and can vary so that each binds to a specific sequence. For example, one second probe can be designed to hybridize to a wild-type sequence, and another second probe can be designed to detect a mutated sequence. Thus, in some instances, a probe set can include one first probe and two second probes (or vice versa).

In some instances, probes can be designed so that they cover conserved regions of a target nucleic acid. Thus, in some instances, a probe (or probe pair can hybridize to similar target nucleic acids in a biological sample (e.g., to detect conserved or similar target nucleic acids) or in different biological samples (e.g., across different species).

In some embodiments, probe sets cover all or nearly all of a genome (e.g., human genome). In some embodiments, probe sets cover all or nearly all of a transcriptome (e.g., human transcriptome). In some embodiments, probe sets cover all or nearly all of a transcriptome for a specific tissue-type or cell-type. In instances where probe sets are designed to cover an entire genome or transcriptome (e.g., the human genome or transcriptome), the methods disclosed herein can detect target nucleic acids in an unbiased manner. In some instances, one probe pair is designed to cover one target nucleic acid (e.g., transcript). In some instances, more than one probe pair (e.g., a probe pair comprising a first probe and a second probe) is designed to cover one target nucleic acid (e.g., transcript). For example, at least two, three, four, five, six, seven, eight, nine, ten, or more probe sets can be used to hybridize to a single target nucleic acid. Factors to consider when designing probes is presence of variants (e.g., SNPs, mutations) or multiple isoforms expressed by a single gene. In some instances, the probe pair does not hybridize to the entire target nucleic acid (e.g., a transcript), but instead the probe pair hybridizes to a portion of the entire target nucleic acid (e.g., transcript).

In some instances, about 50, 100, 200, 300, 400, 500, 600, 700, 800, 900, 1000, 1500, 2000, 2500, 3000, 3500, 4000, 4500, 5000, 10,000, 15,000, 20,000, or more probe pairs (e.g., a probe pair comprising a first probe and a second probe) are used in the methods described herein. In some instances, about 20,000 probe pairs are used in the methods described herein.

In some embodiments, the subset of target nucleic acids includes mRNAs that mediate expression of a set of genes of interest. In some embodiments, the subset of target nucleic acids includes mRNAs that share identical or substantially similar sequences, which mRNAs are translated into polypeptides having similar functional groups or protein domains. In some embodiments, the subset of target nucleic acids includes mRNAs that do not share identical or substantially similar sequences, which mRNAs are translated into proteins that do not share similar functional groups or protein domains. In some embodiments, the subset of target nucleic acids includes mRNAs that are translated into proteins that function in the same or similar biological pathways. In some embodiments, the biological pathways are associated with a pathologic disease. For example, targeted RNA capture can detect genes that are overexpressed or underexpressed in cancer.

In some embodiments, the subset of target nucleic acids includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, or about 1000 target nucleic acids.

In some instances, the methods disclosed herein can detect the abundance and location of at least 5,000, 10,000, 15,000, 20,000, or more different target nucleic acids.

In some embodiments, the subset of target nucleic acids detected by targeted capture methods provided herein includes a large proportion of the transcriptome of one or more cells. For example, the subset of target nucleic acids detected by targeted capture methods provided herein can include at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of the mRNAs present in the transcriptome of one or more cells.

In some instances, the probes are DNA probes. In some instances, the probes are diribo-containing probes.

In some embodiments, the methods described herein include a first probe. As used herein, a “first probe” can refer to a probe that hybridizes to all or a portion of a target nucleic acid and can be ligated to one or more additional probes (e.g., a second probe or a spanning probe). In some embodiments, “first probe” can be used interchangeably with “first probe oligonucleotide.”

In some embodiments, the methods described herein include a second probe. As used herein, a “second probe” can refer to a probe that hybridizes to all or a portion of a target nucleic acid and can be ligated to one or more additional probes (e.g., a first probe). In some embodiments, “second probe” can be used interchangeably with “second probe oligonucleotide.” One of skill in the art will appreciate that the order of the probes is arbitrary, and thus the contents of the first probe and/or second probe as disclosed herein are interchangeable.

In some embodiments, the first and/or second probe includes ribonucleotides, deoxyribonucleotides, and/or synthetic nucleotides that are capable of participating in Watson-Crick type or analogous base pair interactions. In some embodiments, the first and/or second probe includes deoxyribonucleotides. In some embodiments, the first and/or second probe includes deoxyribonucleotides and ribonucleotides. In some embodiments, the first and/or second probe includes a deoxyribonucleic acid that hybridizes to an analyte, and includes a portion of the oligonucleotide that is not a deoxyribonucleic acid. For example, in some embodiments, the portion of the first oligonucleotide that is not a deoxyribonucleic acid is a ribonucleic acid or any other non-deoxyribonucleic acid nucleic acid as described herein. In some embodiments where the first probe includes deoxyribonucleotides, hybridization of the first probe to the mRNA molecule results in a DNA: RNA hybrid. In some embodiments, the first probe includes only deoxyribonucleotides and upon hybridization of the first probe to the mRNA molecule results in a DNA: RNA hybrid.

In some embodiments, the method includes a first and/or second probe that includes one or more sequences that are substantially complementary to one or more sequences of a target nucleic acid. In some embodiments, a first probe includes a sequence that is substantially complementary to a first and/or second target sequence in the nucleic acid. In some embodiments, the sequence of the first and/or second probe that is substantially complementary to the first and/or second target sequence, respectively, in the nucleic acid is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to the first target sequence in the nucleic acid.

In some embodiments, a first and/or second probe includes a sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

In some embodiments, a sequence of the first probe that is substantially complementary to a sequence in the target nucleic acid includes a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides).

In some embodiments, a first probe includes at least two ribonucleic acid bases at the 3′ end. In such cases, a second probe comprises a phosphorylated nucleotide at the 5′ end. In some embodiments, a first probe includes at least three, at least four, at least five, at least six, at least seven, at least eight, at least nine, or at least ten ribonucleic acid bases at the 3′ end.

In some embodiments, the first probe includes one or more barcode sequences selected from a plurality of unique barcode sequences. The barcodes sequences can include degenerate nucleotide sequences or randomized nucleotides sequences. In some embodiments, combinations of one, two, three, or more barcodes sequences are assigned to a first probe sequence that that hybridizes to all or a portion of an analyte, such that the combination of one, two, three, or more barcodes sequences specifically identifies the analyte.

In some embodiments, the one or more barcode sequences of the first probe each include a sequence that is about 5 nucleotides to about 50 nucleotides (e.g., about 5 nucleotides to about 45 nucleotides, about 5 nucleotides to about 40 nucleotides, about 5 nucleotides to about 35 nucleotides, about 5 nucleotides to about 30 nucleotides, about 5 nucleotides to about 25 nucleotides, about 5 nucleotides to about 20 nucleotides, about 5 nucleotides to about 15 nucleotides, about 5 nucleotides to about 10 nucleotides, about 10 nucleotides to about 50 nucleotides, about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 45 nucleotides, about 15 nucleotides to about 40 nucleotides, about 15 nucleotides to about 35 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 25 nucleotides, about 25 nucleotides to about 50 nucleotides, about 25 nucleotides to about 45 nucleotides, about 25 nucleotides to about 40 nucleotides, about 25 nucleotides to about 35 nucleotides, about 25 nucleotides to about 30 nucleotides, about 30 nucleotides to about 50 nucleotides, about 30 nucleotides to about 45 nucleotides, about 30 nucleotides to about 40 nucleotides, about 30 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, or about 45 nucleotides to about 50 nucleotides)

As shown in FIG. 13, a non-limiting example of a first probe 1300, includes a first barcode sequence 1310, a second barcode sequence 1308, and a sequence 1306 that is substantially complementary to a first target sequence in the target nucleic acid. Barcode sequences 1310 and 1308 are selected from a plurality of unique barcode sequences, such that the combination of barcode sequences 1310 and 1308 specifically identifies the target nucleic acid that probe 1300 targets.

In some embodiments, a first probe includes an auxiliary sequence that does not hybridize to a target nucleic acid. In some embodiments, the auxiliary sequence can be used to hybridize to additional probes.

In some embodiments, a second probe includes a capture probe capture domain sequence. As used herein, a “capture probe capture domain” is a sequence, domain, or moiety that can bind specifically to a capture domain of a capture probe. In some embodiments, “capture domain capture domain” can be used interchangeably with “capture probe binding domain.”

In some embodiments, a capture probe capture domain includes a poly(A) sequence. In some embodiments, the capture probe capture domain includes a poly-uridine sequence, a poly-thymidine sequence, or both. In some embodiments, the capture probe capture domain includes a random sequence (e.g., a random hexamer or octamer). In some embodiments, the capture probe capture domain is complementary to a capture domain in a capture probe that detects a particular target(s) of interest. In some embodiments, a capture probe capture domain blocking moiety that interacts with the capture probe capture domain is provided. In some embodiments, a capture probe capture domain blocking moiety includes a sequence that is complementary or substantially complementary to a capture probe capture domain. In some embodiments, a capture probe capture domain blocking moiety prevents the capture probe capture domain from binding the capture probe when present. In some embodiments, a capture probe capture domain blocking moiety is removed prior to binding the capture probe capture domain (e.g., present in a ligation product) to a capture probe. In some embodiments, a capture probe capture domain blocking moiety includes a poly-uridine sequence, a poly-thymidine sequence, or both. In some embodiments, the capture probe capture domain sequence includes ribonucleotides, deoxyribonucleotides, and/or synthetic nucleotides that are capable of participating in Watson-Crick type or analogous base pair interactions. In some embodiments, the capture probe binding domain sequence includes at least 10, 11, 12, 13, 14, 15, 16, 17, 18, 19, or 20 nucleotides. In some embodiments, the capture probe binding domain sequence includes at least 25, 30, or 35 nucleotides.

In some embodiments, a second probe includes a phosphorylated nucleotide at 5′ end. The phosphorylated nucleotide at the 5′ end can be used in a ligation reaction to ligate the second probe to the first probe.

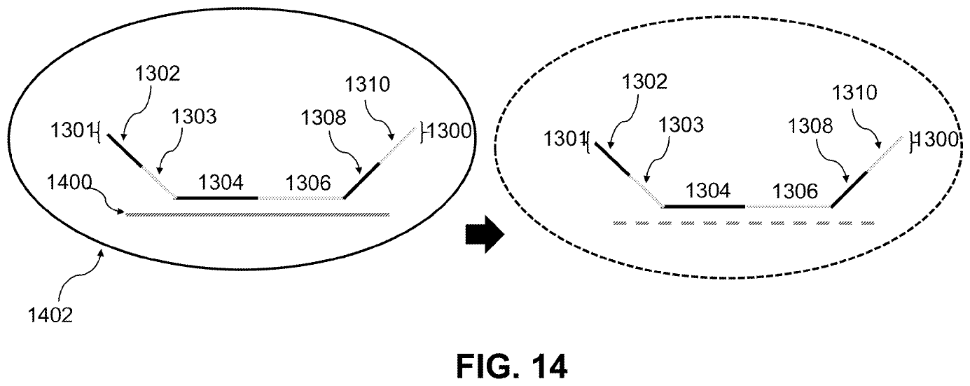

As shown in FIGS. 13 and 14, a non-limiting example of a second probe 1301, includes a first barcode sequence 1303, a sequence 1304 that is substantially complementary to a second target sequence in the target nucleic acid, and a capture probe capture domain 1302 that is complementary to all or a portion of the capture domain of the capture probes affixed to the array. Barcode sequence 1303 is selected from a plurality of unique barcode sequences. Such that the combination of barcode sequences 1310 and 1308 specifically identifies 1402 the target nucleic acid 1400 that probe 1300 targets. After hybridization to the target nucleic acid mediated by sequences 1304 and 1306, a ligation product comprising first probe 1300 and second probe 1301 is generated. The ligation product comprises a unique combination of barcode sequences 1303, 1308, and 1310 that specifically identifies the target nucleic acid that probes 1300 and 1301 both target.

Methods of imaging, sample preparation, probe hybridization, washing, ligation, permeabilization, and blocking have been described previously in WO 2021/133849, US 2021/0348221, and US 2021/0285046, each of which is incorporated by reference in its entirety.

### III Biological Samples

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some instances, the biological sample can include one or more diseased cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells,

In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probes are added. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

### IV. Kits

In some embodiments, also provided herein are kits that include one or more reagents to detect one or more analytes described herein. In some instances, the kit includes a substrate comprising a plurality of capture probes comprising a capture domain. In some instances, the kit includes a plurality of probes (e.g., a first probe and a second probe, each comprising one or more unique barcode sequences).

A non-limiting example of a kit used to perform any of the methods described herein includes: (a) a substrate comprising a plurality of capture probes a capture domain; (b) a system comprising: a plurality of first probes and second probes, wherein a first probe and a second probe each comprises sequences that are substantially complementary to a target nucleic acid and one or more unique barcode sequences, and wherein the second probe comprises a capture binding domain; and (c) instructions for performing any of the methods disclosed herein.

Another non-limiting example of a kit used to perform any of the methods described herein includes: (a) an array comprising a plurality of capture probes; (b) a plurality of probes comprising a first probe and a second, wherein the first probe and the second probe are substantially complementary to adjacent sequences of a target nucleic acid and each comprise one or more unique barcode sequences, wherein the second probe comprises (i) a capture probe binding domain that is capable of binding to a capture domain of the capture probe and (ii) a linker sequence; (c) a plurality of enzymes comprising a ribonuclease and a ligase; and (d) instructions for performing any of the methods disclosed herein.

Another non-limiting example of a kit used to perform any of the methods described herein includes: (a) an array comprising a plurality of capture probes; (b) a plurality of probes comprising a first probe and a second probe, wherein the first probe and the second probe are substantially complementary to adjacent sequences of a target nucleic acid and each comprise one or more unique barcode sequences, wherein the first probe includes a linker sequence, wherein the second probe comprises a capture probe binding domain that is capable of binding to a capture domain of the capture probe; (c) a plurality of enzymes comprising a ribonuclease and a ligase; and (d) instructions for performing any of the methods disclosed herein.

In some embodiments of any of the kits described herein, the kit includes a second probe that includes a preadenylated phosphate group at its 5′ end and a first probe comprising at least two ribonucleic acid bases at the 3′ end.

### EXAMPLES

### Example 1. Detection of Analytes Using Templated Ligation Probes Comprising Barcodes and Nanoparticle-Conjugated Probes

This example illustrates a method of analyzing a biological sample by generating spatial information of target nucleic acid molecules using templated ligation with barcoded probes followed by 2-bit combinatorial barcode detection by electron microscopy as a readout.

A fresh frozen mouse brain section is placed on a glass slide array which is covered by a plurality of poly(T) capture probes. The section is fixed with formaldehyde and permeabilized with pepsin and HCl. The tissue section is treated with pre-hybridization buffer (e.g., hybridization buffer without the first and second probes), a reagent solution comprising pairs (e.g., hundreds or thousands of pairs) of barcoded templated ligation probes targeting a panel of mRNA sequences is applied to the tissue section. Templated ligation probe pairs are designed to hybridize adjacent sequences of target mRNA sequences, each templated ligation probe comprises a plurality (e.g., one, two, three or more) of unique barcode sequences such that each pair of templated ligation probes comprise a unique combination of barcode sequences specifically identifying a single mRNA target sequence. One templated ligation probe of each pair of probes also includes a capture domain, such as a poly(A) capture domain, that can hybridize to a complementary capture domain of a capture probe on an array.

Templated ligation probes are hybridized to target mRNA nucleic acids within the tissue section, and the array is washed. Ligase is added to the samples to ligate hybridized probes to generate ligation products. Probes are released from the mRNA targets by contacting the tissue section with RNAse H. Samples are permeabilized to facilitate capture of the ligation product by the capture probes on the array. Ligation products that hybridize to the capture probes are extended using a polymerase. Following extension, the ligation product is released or dehybridized from the extended capture probe.

In order to spatially detect extended capture probes on the array, wherein the extended capture probes correspond to ligation products that serve as proxies for the target mRNA within the tissue section, successive rounds of hybridization and imaging of conjugated decoding primers are performed. Each conjugated decoding primer includes a gold nanoparticle approximately 5 nM in diameter, and the primer sequence specifically hybridizes to a barcode sequence within an extended capture probe (now containing the barcode sequence or a complement thereof). After each cycle of hybridization of a decoding primer, the array is imaged in a single plane by interferometric cross-polarization microscopy at ultralow excitation powers. Where a decoding primer hybridizes to a barcode within an extended capture probe, the conjugated gold nanoparticle is detected and recorded in a spatial image of the array. As a result, only subsets of extended capture probes corresponding to target mRNA sequences are detected during each cycle. After each cycle of hybridization and detection, hybridized decoding primers are de-hybridized and washed from the array. After successive rounds of hybridization and detection of a series of conjugated decoding primers, a 2-bit signature for each extended capture probe on the array is generated, with each 2-bit signature uniquely identifying the target analyte corresponding to a pair of ligation products (see, e.g., FIG. 23). Using this method, the spatial location of a plurality of target mRNA sequences within the tissue section is identified and determined based on the unique combination of the one or more barcodes of the first probe and the one or more barcodes of the second probe.

### Example 2 Detection of Analytes Using Templated Ligation Probes Comprising Barcodes and Fluorescence

This example illustrates a method of analyzing a biological sample by generating spatial information of nucleic acid molecules using templated ligation with barcoded probes followed by detection and decoding using oligonucleotide decoding primers and incorporation of fluorescently labeled nucleotides with fluorescent microscopy imaging as a readout.

A fresh frozen mouse brain section is placed on a glass slide array which is covered by a plurality of capture probes comprising a poly(T) capture sequence. The section is fixed with formaldehyde, then permeabilized with pepsin and HCl. The tissue section is treated with pre-hybridization buffer (e.g., hybridization buffer without the first and second probes), which is a reagent solution comprising barcoded probes targeting a panel of mRNA target sequences. Probes are designed to hybridize to adjacent sequences of each mRNA target, and each probe comprises unique barcode sequences (e.g., one, two, three, or more barcodes) such that each pair of probes comprises a unique combination of barcode sequences specifically identifying an mRNA target. One probe of each pair of probes also includes a capture domain, for example a poly(A) capture domain. Probes are hybridized to specific target mRNA sequences within the tissue section, and the array is washed. Ligase is added to the samples to ligate hybridized probes to generate ligation products. Probes are released from the mRNA targets using RNAse H. Samples are permeabilized to facilitate capture of the ligation product by the capture probes on the array. Ligation products that hybridize to the capture probes are extended and the ligation products are released or dehybridized from the extended capture probes.

In order to spatially detect extended capture probes on the array corresponding to ligation products, which in turn serve as proxies for the target mRNA, within the tissue section, successive rounds of hybridization of decoding primers followed by primer extension and incorporation of fluorescently labeled nucleotides with fluorescent microscopy imaging as a readout is performed. Each cycle of decoding includes, for example, four sequential reactions for identifying the four nucleotides. Each reaction results in the labeling of one extended capture probe or set of extended capture probes with a fluorophore labelled nucleotide. Each cycle of decoding the extended capture probes on the array includes four rounds of hybridization of decoding primers (e.g. decoding primers 1.1, 1.2, 1.3, 1.4), extension of the decoding primers by a polymerase and fluorescently labeled nucleotides using the extended capture probes as templates, and imaging by fluorescent microscopy.

For a single round of decoding by fluorescent microscopy, a barcode-specific decoding primer is hybridized to one of the barcode sequences of an extended capture probe (see, e.g., FIG. 17). A reagent mix including a polymerase and a mixture of dNTPs is applied to the array, for example with the dATPs of the mix labeled with a fluorophore. During extension from the decoding primer, the labeled dATPs are incorporated into the extension product. Because the capture probes of the array include a poly(T) sequence (in this example), an abundance of fluorescently labeled dATPs are incorporated into the extension product, enabling detection by fluorescent microscopy. After the first round of hybridization and extension, excess polymerase and nucleotides are washed from the array.

For a second round of decoding the extended capture probe, a second barcode-specific decoding primer is hybridized to a different barcode sequence of an extended capture probe (see, e.g., FIG. 18). A reagent mix including a polymerase and a mixture of dNTPs is applied to the array, with the dATPs of the mix labeled by a different fluorophore that the first round of decoding. During extension from the decoding primer, the labeled dATPs are incorporated into the extension product. Because the capture probes of the array include a poly(T) sequence, in this example, an abundance of fluorescently labeled dATPs are incorporated into the extension product, enabling detection of the second fluorescent moiety by fluorescent microscopy. After the second round of hybridization and extension, excess polymerase and nucleotides are washed from the array.

A third round and a fourth round of hybridization and extension are performed, using a third and a fourth different fluorophore attached to dATP (see, e.g., FIGS. 19 and 20). After one cycle of four rounds of hybridization and extension using four different fluorophores, the extended capture probes are decorated with 20-50 fluorophores each (for example). The array is imaged by fluorescent microscopy, spatially recording the location and identity of extended capture probes decorated with each of the four fluorophores. After imaging, decoding primer extension products are de-hybridized and a new cycle of four rounds of hybridization and extension is performed. Successive cycles of decoding, with four rounds of hybridization and extension for each cycle, are performed to resolve the identity and spatial location of potentially hundreds or thousands of target mRNAs in the experiment. Using this method, the spatial location of a plurality of target mRNAs within the tissue section is identified and determined based on the unique combination of the one or more barcodes of the first probe and the one or more barcodes of the second probe.

1. A method for determining a location of a target nucleic acid in a biological sample, the method comprising:
(a) providing the biological sample on a first substrate;
(b) contacting a first probe and a second probe with the biological sample, wherein the first probe and the second probe each comprise sequences that are substantially complementary to sequences of the target nucleic acid, wherein the first probe or the second probe comprises a capture probe capture domain that is complementary to all or a portion of a capture domain on an array, wherein the first probe and/or the second probe comprises one or more barcode sequences selected from a plurality of barcode sequences, such that a combination of the first probe and the second probe comprises a unique combination of the one or more barcode sequences;
(c) hybridizing the first probe and the second probe to the target nucleic acid;
(d) generating a ligation product by ligating the first probe and the second probe;
(e) releasing the ligation product from the target nucleic acid;
(f) hybridizing the ligation product to the capture domain on the array comprising a plurality of capture probes, wherein a capture probe of the plurality of capture probes comprises a capture domain; and
(g) decoding the one or more barcode sequences or complements thereof, and using the decoded one or more barcode sequences to determine the location of the target nucleic acid in the biological sample.

2-3. (canceled)

4. The method of claim 1, wherein the ligation product comprises the combination of the one or more barcode sequences.

5. The method of claim 2, wherein the combination of the one or more barcode sequences identifies the target nucleic acid.

6. The method of claim 1, wherein:
(a) the first probe comprises, in order from 5′ to 3′:
(i) a functional sequence,
(ii) the one or more barcode sequences, and
(iii) the sequence substantially complementary to at least a portion of the sequence of the target nucleic acid; and/or
(b) the second probe comprises, in order from 5′ to 3′:
(i) the sequence substantially complementary to at least a portion of the sequence of the target nucleic acid,
(ii) the one or more barcode sequences, and
(iii) the capture probe capture domain.

7. The method of claim 1, wherein the decoding step further comprises:
(i) at step (f), extending the capture domain using the ligation product as a template;
(ii) hybridizing a first primer to a first barcode sequence of the plurality of barcode sequences of the extended capture domain, wherein the first primer is conjugated to a labeling agent, and wherein the first primer comprises a sequence substantially complementary to the first barcode sequence;
(iii) detecting hybridization of the first primer to the first barcode sequence;
(iv) removing the first primer from the first barcode sequence; and
(v) repeating steps (ii) through (v) using a second, a third, a fourth, a fifth, or more primers, each comprising the labeling agent, to detect a second, a third, a fourth, a fifth, or more barcode sequences, respectively.

8. The method of claim 7, wherein the labeling agent is a bead, a nanoparticle, or a gold nanoparticle.

9-10. (canceled)

11. The method of claim 7, wherein the detecting hybridization of the first primer comprises imaging the array.

12-13. (canceled)

14. The method of claim 1, wherein the decoding step further comprises:
(i) at step (f), extending the capture domain using the ligation product as a template,
(ii) hybridizing a first primer to a first barcode sequence of the plurality of barcode sequences of the extended capture domain, wherein the first primer comprises a sequence substantially complementary to the first barcode sequence;
(iii) applying a mixture of dNTPs and a polymerase to the array;
(iv) extending the first primer to produce a first extension product;
(v) detecting the first extension product; and
(vi) repeating steps (ii) through (v) using a second, a third, a fourth, a fifth, or more primers to detect a second, a third, a fourth, a fifth, or more extension products, respectively.

15. The method of claim 14, wherein each of the first, the second, the third, the fourth, the fifth, or more primers hybridize to unique barcode sequences of the plurality of barcodes sequences.

16. The method of claim 14, wherein the mixture of dNTPs comprises dATP, dTTP, dCTP, and dGTP and wherein one of dATP, dTTP, dCTP or dGTP is labeled with a fluorophore.

17. The method of claim 16, wherein for each cycle of steps (ii) through (v), the dATP, dTTP, dCTP or dGTP is labeled with a unique fluorophore, and wherein the detecting comprises imaging the array.

18-19. (canceled)

20. The method of claim 14, wherein the first primer is selected from a set of four primers, each primer having a different 3′-terminal nucleotide, the first primer having a 3′-terminal nucleotide complementary to the first 5′ nucleotide of the sequence that is substantially complementary to the sequence of the target nucleic acid.

21-41. (canceled)

42. The method of claim 1, wherein the capture domain comprises a spatial barcode.

43. The method of claim 1, wherein the biological sample is a tissue sample or a cell culture sample.

44-52. (canceled)

53. The method of claim 1, wherein the array is on the first substrate.

54. The method of claim 1, wherein the array is on a second substrate, wherein the first substrate is aligned with the second substrate, such that at least a portion of the biological sample is aligned with at least a portion of the array.

55. (canceled)

56. A composition comprising:
(a) an array comprising capture probes, wherein the capture probes comprise a capture domain;
(b) a biological sample on the array, wherein the biological sample comprises a plurality of target nucleic acids of interest; and
(c) a first probe and a second probe hybridized to a target nucleic acid and ligated together, wherein the first probe and the second probe each comprise a sequence that is substantially complementary to adjacent sequences of the target nucleic acid, wherein the first probe and/or the second probe contain at least two unique barcode sequences selected from a plurality of barcode sequences, and wherein one of the first probe or the second probe comprises a capture probe capture domain.

57. The composition of claim 56, wherein the first probe and second probe are ligated together and collectively comprise a unique combination of barcode sequences that identify an analyte.

58. The composition of claim 56, further comprising a first primer that is hybridized to one of the barcode sequences, wherein the first primer is conjugated to a labeling agent.

59. The composition of claim 56, further comprising a first primer that is hybridized to one of the barcode sequences, and a mixture of dNTPs, wherein the mixture of dNTPs comprises at least one dNTP that is labeled with a fluorophore.
